(19) United States
(12) Patent Application Publication
(10) Pub. No.: US 2022/0177666 A1
(43) Pub. Date: Jun. 9, 2022

(54) MOLECULARLY IMPRINTED POLYMERS FOR EXTRACTION OF CANNABINOIDS AND USES THEREOF

(71) Applicant: 6TH Wave Innovations Corp., Baltimore, MD (US)

(72) Inventors: Aristotle Kalivretenos, Ellicott City, MD (US); Louis Reichel, Ellicott City, MD (US); Jonathan Gluckman, Arnold, MD (US)

(21) Appl. No.: 17/438,343

(22) PCT Filed: Mar. 12, 2020
(86) PCT No.: PCT/US2020/022427

§ 371 Date: Sep. 10, 2021

Related U.S. Application Data
(60) Provisional application No. 62/817,100, filed on Mar. 12, 2019.

(51) Int. Cl. C08J 9/28; C07D 311/80; C07C 37/68; C07C 69/88; B01J 20/26; B01D 11/04

(52) U.S. Cl. CPC C08J 9/286; C07D 311/80; C07C 37/685; C07C 69/88; B01J 2220/4812; B01D 11/0492; C07C 2601/14; C07C 2601/16; C08J 2325/08; B01J 20/264

(57) ABSTRACT

The present disclosure relates to molecularly imprinted polymers that target cannabinoid(s), including THC and CBD, as well as methods of making molecularly imprinted polymers that target cannabinoid(s), including THC and CBD and uses thereof.

### CROSS-REFERENCE TO RELATED APPLICATIONS

The present application claims the benefit of priority to U.S. Provisional Application No. 62/817,100, filed on Mar. 12, 2019, the contents of which is hereby incorporated by reference in its entirety.

### BACKGROUND OF THE DISCLOSURE

“Cannabis” refers to a genus of flowering plants that include the species, Cannabis sativa, Cannabis indica, and Cannabis ruderalis. The Cannabis industry has flourished as attitudes towards medicinal and recreational use of Cannabis continue to evolve. In the United States, a growing number of states have approved the medicinal and recreational use of marijuana. Canada legalized recreational use country wide in 2018. This has led to an increase in growers, processers and the need for analytical characterization and regulation of products. Processed materials must be evaluated for the active Cannabis components as well as potential harmful impurities (e.g. pesticides, heavy metals and toxins). This includes Cannabis for smoking as well as other applications utilizing enriched or purified extracts of the cannabinoid components.

Cannabis is composed of many chemical compounds, including cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. The cannabinoids cannabidiol (“CBD”, which is non-psychoactive) and the Δ-9-tetrahydrocannabinol (“THC”, which is psychoactive) are of particular importance. The availability of purified CBD and THC allows for controlled specialty Cannabis extract formulations for different medicinal indications and/or recreational uses.

A highly efficient, commercially viable, low cost purification process for CBD and THC from crude Cannabis extracts would be of great value. Applicability to extraction from hemp would provide added value and increase the growing options to meet increasing demand.

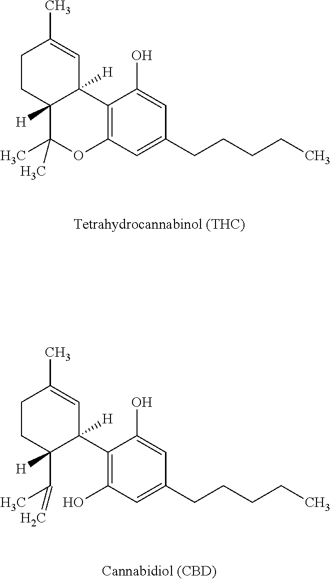 [SMILES: CCCCCc1cc(O)c2c(c1)OC(C)(C)[C@@H]1CCC(C)=C[C@@H]21 ; C=C(C)[C@@H]1CCC(C)=C[C@H]1c1c(O)cc(CCCCC)cc1O]

The current methods to generate pure THC and CBD from a mixture involve chromatography, typically as the final purification of pre-processed material (i.e., material where all components except cannabinoids are removed). Chromatography suffers from various shortcomings that limit its commercial viability.

As such, there is a need for economical methods of isolating purified cannabinoids (such as THC and CBD) from mixtures.

### SUMMARY OF THE DISCLOSURE

The present disclosure relates to molecularly imprinted polymers. More particularly, the disclosure relates materials comprising molecularly imprinted polymers that target cannabinoids, including THC and CBD.

In some embodiments, the present disclosure provides macroreticular polymer beads and methods of making and using the same. The present disclosure also provides methods for the selective isolation and purification of cannabinoids from crude extracts with minimal processing. The disclosure addresses a need for new technologies for a commercially viable process for the production of pure cannabinoid compounds for medicinal and recreational applications.

In one aspect, the present disclosure provides a plurality of macroreticular polymer beads comprising a copolymer having a plurality of complexing cavities that selectively bind a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid and
wherein the cannabinoid surrogate is removed to provide a complexing cavity that selectively binds a target cannabinoid.

In one aspect, the present disclosure provides a plurality of macroreticular polymer beads comprising a copolymer that selectively binds a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid.

In one aspect, the disclosure provides a plurality of macroreticular polymer beads comprising a copolymer having a plurality of complexing cavities which selectively bind a target cannabinoid, wherein the copolymer is prepared from:
(a) a cannabinoid surrogate,
(b) a non-crosslinking monomer, and
(c) a crosslinking monomer:
wherein the cannabinoid surrogate has substantially the same steric and functional properties of the target cannabinoid and
wherein the cannabinoid surrogate is removed to provide a complexing cavity that selectively binds a target cannabinoid.

In one aspect, the present disclosure provides methods of preparing macroreticular molecularly imprinted polymer beads comprising:
(1) polymerizing:
   (a) a cannabinoid surrogate-containing monomer,
   (b) a non-crosslinking monomer, and
   (c) a crosslinking monomer;
   wherein the cannabinoid surrogate has substantially the same steric and functional properties of the target cannabinoid and
(2) removing the cannabinoid surrogate to provide a complexing cavity that selectively binds a target cannabinoid.

In one aspect, the present disclosure provides methods of preparing a macroreticular molecularly imprinted polymer that selectively binds a target cannabinoid, the method comprising:
(1) polymerizing:
   (a) a cannabinoid surrogate-containing monomer,
   (b) a non-crosslinking monomer, and
   (c) a crosslinking monomer;
wherein the cannabinoid surrogate has substantially the same steric and functional properties of the target cannabinoid.

In one aspect, the present disclosure provides methods of preparing macroreticular molecularly imprinted polymer beads, the method comprising:
(1) polymerizing:
   (a) cannabinoid surrogate,
   (b) a non-crosslinking monomer, and
   (c) a crosslinking monomer:
   wherein the cannabinoid surrogate has substantially the same steric and functional properties of the target cannabinoid and
(2) removing the cannabinoid surrogate to provide a complexing cavity that selectively binds a target cannabinoid.

Some embodiments relate to a method of selectively sequestering one or more target cannabinoids from a solution of the one or more target cannabinoids admixed with other Cannabis extract components, comprising first contacting the macroreticular polymer beads with a stripping solution, whereby the cannabinoid surrogates are removed from the macroreticular polymer beads, then contacting the stripped beads with the solution, thereby selectively sequestering the target cannabinoid in the macroreticular polymer beads. In some embodiments, the solution is a crude or semi-processed Cannabis extract. In some embodiments, the solution is a crude or semi-processed hemp plant extract.

### DETAILED DESCRIPTION OF THE DRAWINGS

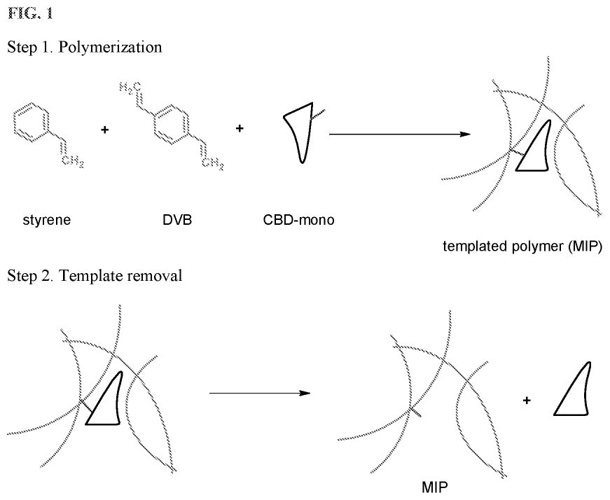

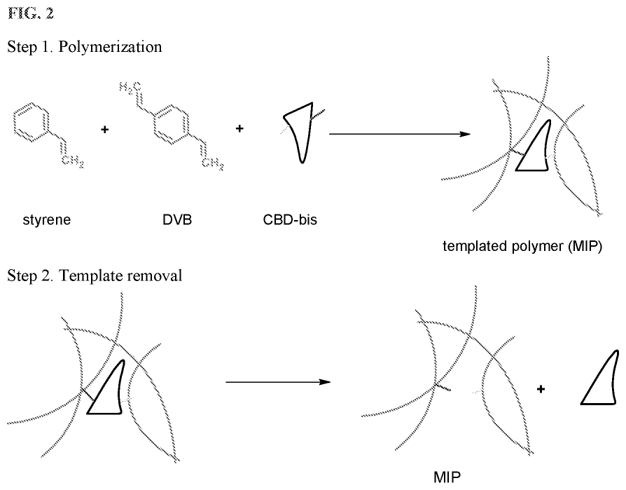

FIG. 1 shows a method of preparing a molecular imprinted polymer (MIP) of the present disclosure using a mono-covalent surrogate-containing monomer (CBD-mono).

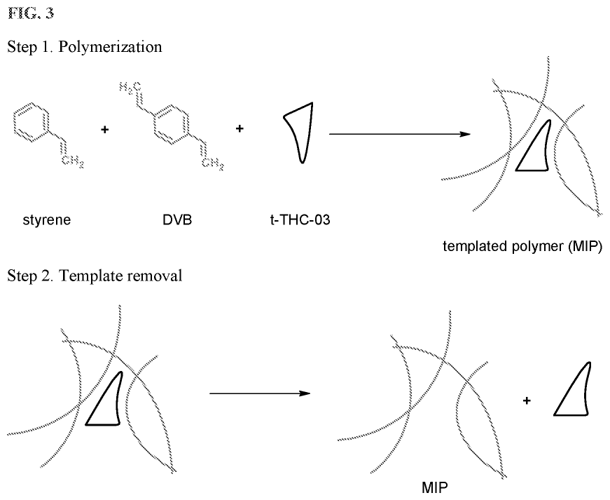

FIG. 2 shows a MIP of the present disclosure prepared using a bis-covalent surrogate-containing monomer (CBD-bis).

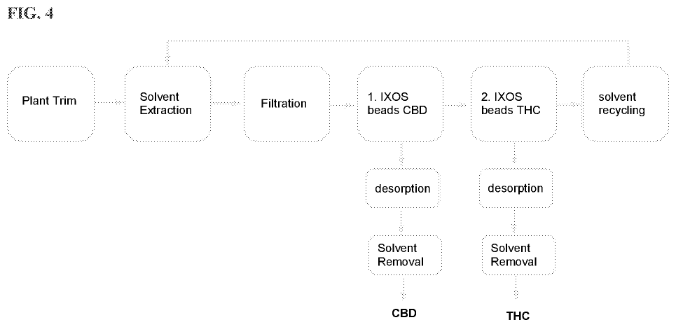

FIG. 3 shows a MIP of the present disclosure prepared using a non-covalent surrogate (t-THC-03).

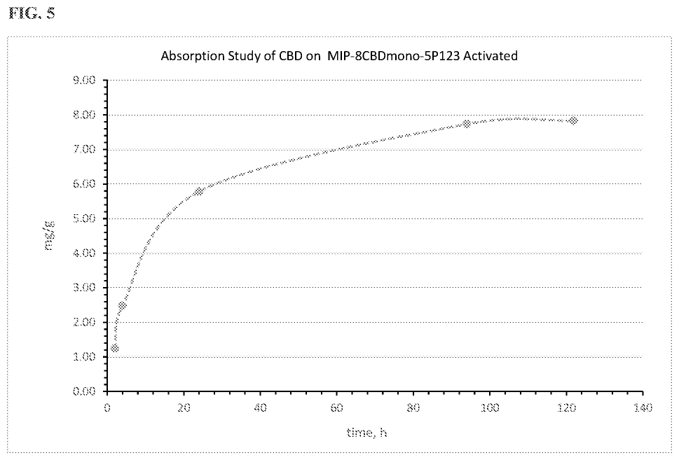

FIG. 4 shows the use of the materials of the present disclosure to isolate CBD/THC.

FIG. 5 shows absorption of CBD on KOH activated CBD-mono MIP beads.

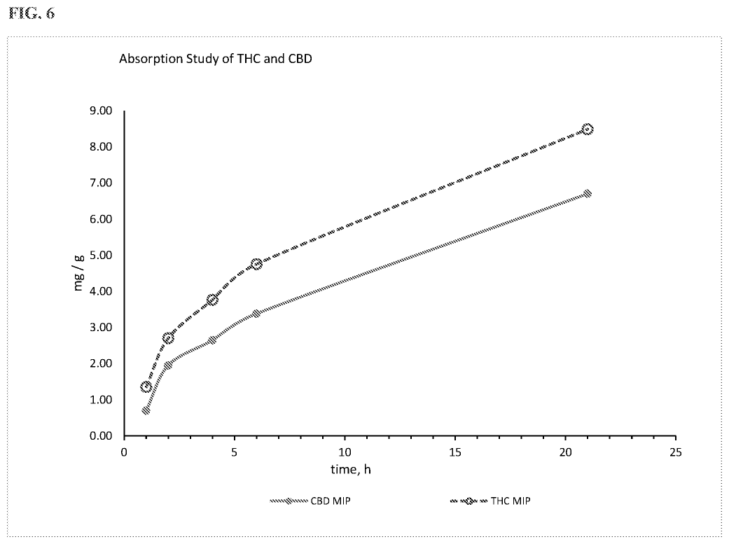

FIG. 6 shows the absorption of CBD and THC on unactivated CBD-mono MIP beads.

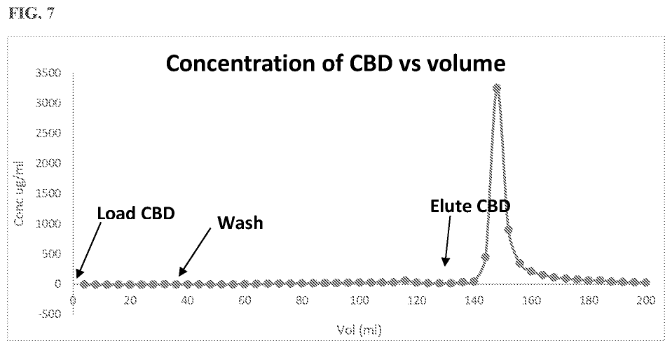

FIG. 7 depicts a UV/Vis trace showing elution of CBD from unactivated CBD-mono MIP beads.

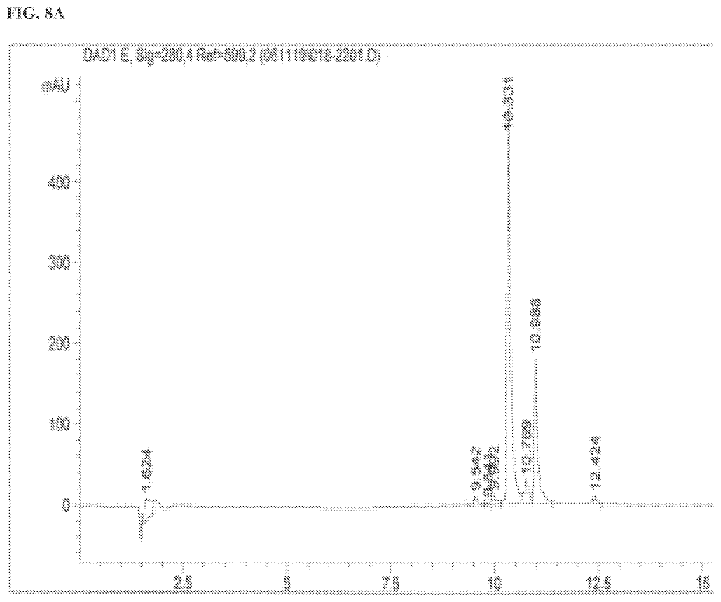

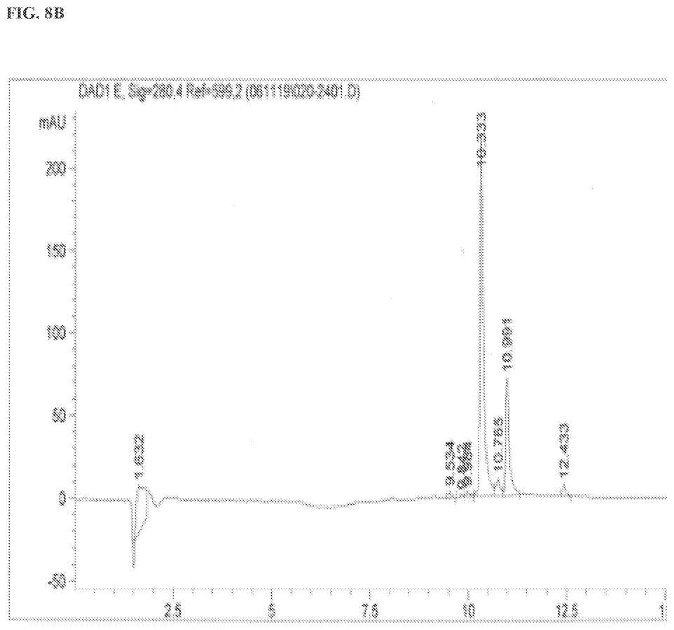

FIG. 8A shows the RP-HPLC chromatogram (280 nm) of CBD/THC 4:1 stock solution.

FIG. 8B shows the RP-HPLC chromatogram (280 nm) of pooled ethanol elution fractions.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The term “Cannabis plant(s)” encompasses wild type Cannabis sativa and also variants thereof, including Cannabis chemovars which naturally contain different amounts of the individual cannabinoids, Cannabis sativa subspecies indica including the variants var. indica and var. kafiristanica, Cannabis indica and also plants which are the result of genetic crosses, selfcrosses or hybrids thereof. The term “Cannabis plant material” is to be interpreted accordingly as encompassing plant material derived from one or more Cannabis plants. For the avoidance of doubt it is hereby stated that “Cannabis plant material” includes dried Cannabis biomass.

As used herein, the term “chemovar” means plants distinguished by the chemical compounds produced, rather than the morphological characteristics of the plant.

As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

The term “plant material” encompasses a plant or plant part (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Centre for Drug Evaluation and Research.

The term “enriched” or “enriched target cannabinoids(s)” means preparations of any one of the target cannabinoid(s) having a chromatographic purity (e.g., as determined by area normalization of an HPLC profile) of the target cannabinoid of greater than about 80%. In some embodiments, the chromatographic purity is greater than 85%. In some embodiments, the chromatographic purity is greater than about 90%. An enriched preparation of target cannabinoid(s) will generally contain a greater proportion of impurities and/or other cannabinoids than a substantially pure preparation of the same target cannabinoids(s), as described below.

The term “substantially pure” or “substantially pure target cannabinoid(s)” means preparations of any one of the target cannabinoid(s) having a chromatographic purity of the target cannabinoid(s) of greater than about 95% (e.g., as determined by area normalization of an HPLC profile). In some embodiments, the chromatographic purity is greater than 96%. In some embodiments, the chromatographic purity is greater than about 97%. In some embodiments, the chromatographic purity is greater than about 98%. In some embodiments, the chromatographic purity is greater than about 99%. In some embodiments, the chromatographic purity is greater than about 99.5%.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R_{b} is an alkylene, alkenylene, or alkynylene group as defined above and R_{d} is a carbocyclyl radical as defined above. Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized, the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups, a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced

with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g}, and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

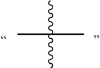

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

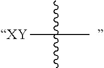

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

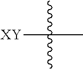

infers that when R³ is “XY”, the point of attachment bond is the same bond as the bond by which R³ is depicted as being bonded to CH₃.

### DETAILED DESCRIPTION OF THE DISCLOSURE

“Cannabis” refers to a genus of flowering plants that include the species, Cannabis sativa, Cannabis indica, and Cannabis ruderalis. The Cannabis industry has flourished as attitudes towards medicinal and recreational use of Cannabis continue to evolve. In the United States, a growing number of states have approved the medicinal and recreational use of marijuana. Canada legalized recreational use country wide in 2018. This has led to an increase in growers, processers and the need for analytical characterization and regulation of products. Processed materials must be evaluated for the active Cannabis components as well as potential harmful impurities (e.g. pesticides, heavy metals and toxins). This includes Cannabis for smoking as well as other applications utilizing enriched or pure extracts of the cannabinoid components.

Cannabis is composed of many chemical compounds, including cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. The cannabinoids cannabidiol (“CBD”, which is non-psychoactive) and the Δ-9-tetrahydrocannabinol (“THC”, which is psychoactive) are of particular importance. The availability of pure CBD and THC allows for controlled specialty Cannabis extract formulations for different medicinal indications and/or recreational uses.

A highly efficient, commercially viable, low cost purification process for CBD and THC from crude Cannabis extracts would be of great value. Applicability to extraction from hemp would provide added value and increase the growing options to meet increasing demand.

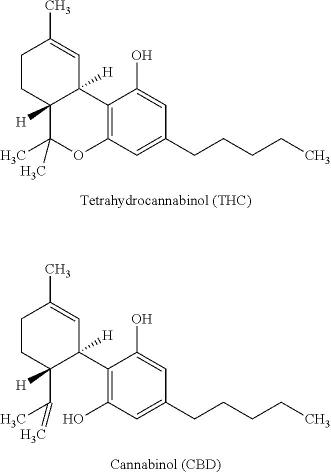

The current methods to generate pure THC and CBD from a mixture involve chromatography, typically as the final purification of pre-processed material (i.e., material where all components except cannabinoids are removed). Chromatography suffers from various shortcomings that limit its commercial viability.

As such, there is a need for economical methods of isolating purified cannabinoids (such as THC and CBD) from mixtures.

Several methods for isolating and purifying CBD and THC from Cannabis extracts, include chromatography, extraction with organic solvents (e.g. butane), sub- and supercritical CO₂ extraction and distillation. U.S. Pat. No. 9,987,567 by NextLeaf discloses a process that combines several of these processes for the isolation of cannabinoids. More recently, the use of membrane extraction has been reported by Green Sky Laboratories (Green Sky Labs, Poster Presentation, March 2018).

In general, the extraction processes using organic solvents (e.g. propane, butane) and sub- and supercritical CO₂ result in mixtures of cannabinoids and can also contain terpenes/waxes depending on post-extraction processing. The Green Sky process also falls into this category. Although it is claimed to provide pure CBD or THC, it does not perform any separation of cannabinoids. The claims stated come from the use of hemp (for CBD) and marijuana plant (for THC) as starting materials, which assumes they only contain CBD and THC, respectively. These processes may be used as a pre-processing step to yield material for further purification to produce pure THC and CBD (e.g. provide pre-purified material for chromatography).

The use of supercritical CO₂ typically provides low yield (throughput) and does not separate all compounds away from THC and CBD (e.g. other terpenes and waxes). Moreover, there are explosion hazards involved utilizing high pressure and temperature solvents. Solvent extractions also suffer from lack of selectivity and potential toxicity and flammability hazards based on the solvents utilized.

Currently, the only methods to generate pure THC and CBD from a mixture utilize chromatography. Chromatography is typically a final processing step with pre-processed material (i.e., a material where all components except cannabinoids are removed) as the starting material, adding cost to the final pure products.

Chromatography suffers from various shortcomings that limit its commercial viability. For example, chromatography is a labor-intensive process, often provides incomplete purification (i.e., some residual impurities/undesired side-products persist in the chromatographed material) and generally low yielding. Thus, chromatography is not cost effective and is limited in scale. Chromatography also requires relatively pure cannabinoid material (e.g. terpenes and waxes removed) to improve efficiency and limit fouling of chromatography media (e.g. silica gel). Depending on the solvent system used, there are also cost concerns and safety concerns such as explosion hazards (CO₂ solvent), potential toxicity and flammability hazards.

In general, the deficiencies of current technologies limit their commercial viability due to cost, insufficient product purity and low throughput/recovery, as well as limited scalability.

Thus, efficient, commercially viable purification processes for cannabinoids, (in particular, CBD and THC) from crude Cannabis extracts are needed.

The use of molecular imprinted polymers (MIPs) is a proven technique for highly selective extraction of desired target molecules from complex mixtures. In general, a polymer is prepared with defined rigid pockets designed to only allow the target molecule to fit in and be trapped. The pockets are prepared by including the target molecule or a similar compound as a template during the polymerization process, which is removed to provide the active MIP.

In practice, a mixture of the target molecule and impurities is added to the polymer, and the target molecule is selectively trapped in the pockets. After washing away all other untrapped materials, the purified target is removed from the polymer. Several groups have reported the preparation of MIPs using a precipitation polymerization process with reported affinity and selectivity for CBD and THC, although they utilize THC (or metabolites) as the template which is not commercially viable. Another report utilized a non-cannabinoid template (catechin, a flavonoid) in a similar manner for THC/CBD extraction (Anal. Bioanal. Chem. (2014) 406(15), 3589-97).

MIPs are polymers designed to be highly selective for a specific target molecule. MIPs are prepared by polymerizing a polymerizable ligand which coordinates or “binds” to the target molecule. The target molecule and the polymerizable ligand are incorporated into a pre-polymerization mixture, allowed to form a complex, then polymerized (typically in the presence of one or more non-crosslinking monomers and a cross-linking monomer) to form a complexed. The target molecule thus acts as a “template” to define a cavity or absorption site within the polymerized matrix which is specific to the target molecule (e.g., has a shape or size corresponding to the target molecule). The target molecule is then removed from the MIP prior to its use as an absorbent.

The preparation and use of MIPs for the isolation of organic ions utilizing macroreticular polymer beads (IXOS™ beads) is described in U.S. Pat. No. 9,504,988. The IXOS™ bead technology has proven to be robust and reproducible in gold mining applications, utilized in a continuous feed manner using a carousel type system.

MIPs are polymers designed to be highly selective for a specific target molecule. MIPs are prepared by polymerizing a polymerizable ligand which coordinates or “binds” to the target molecule. The target molecule and the polymerizable ligand are incorporated into a pre-polymerization mixture, allowed to form a complex, and then polymerized (typically in the presence of one or more non-crosslinking monomers and a cross-linking monomer). The target molecule thus acts as a “template” to define a cavity or absorption site within the polymerized matrix which is specific to the target molecule (e.g., has a shape or size corresponding to the target molecule). The target molecule is then removed from the MIP prior to its use as an absorbent.

The present disclosure provides materials comprising MIPs for the efficient, commercially viable purification processes for cannabinoids (in particular, CBD and THC) from crude Cannabis extracts.

### Target Molecules:

The present disclosure provides materials and methods for separating, extracting, or sequestering a specific component of a Cannabis extract, such as tetrahydrocannabinol (THC) and cannabidiol (CBD), from a mixture. In the context of the present disclosure, the target molecule is generally a cannabinoid (such as THC or CBD). In some embodiments, the target molecule is more than one cannabinoid (e.g., the MIP sequesters THC and CBD). In some embodiments, the target molecule is multiple cannabinoids present in a mixture (e.g., the MIP sequesters the multiple cannabinoids present in a Cannabis or hemp extract).

In some embodiments, the target cannabinoid is selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄(CBD-C₄), cannabidivarnic acid (CBDVA), cannabidivarin (CBDV), cannabidiorcol (CBD-C₁), tetrahydrocannabinolic acid A (THCA-A), tetrahydrocannabinolic acid B (THCA-B), tetrahydrocannabinolic acid (THCA), tetrahydrocannabinol (THC), tetrahydrocannabinolic acid C₄(THCA-C₄), tetrahydrocannabinol C₄ (THC-C₄), tetrahydrocannabivarinic acid (THCVA), tetrahydrocannabivarin (THCV), tetrahydrocannabiorcolic acid (THCA-C₁), tetrahydrocannabiorcol (THC-C₁), Δ⁷-cis-iso-tetrahydrocannabivarin, Δ⁸-tetrahydrocannabinolic acid (Δ⁸-THCA), cannabivarinodiolic (CBNDVA), cannabivarinodiol (CBNDV), Δ⁸-tetrahydrocannabinol (Δ⁸-THC), Δ⁹-tetrahydrocannabinol (Δ⁹-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabivarinselsoin (CBEV), cannabivarinselsoinic acid (CBEVA), cannabielsoic acid (CBEA), cannabielvarinsoin (CBLV), cannabielvarinsoinic acid (CBLVA), cannabinolic acid (CBNA), cannabinol (CBN), cannabivarinic acid (CBNVA), cannabinol methylether (CBNM), cannabinol-C₄(CBN-C₄), cannabivarin (CBV), cannabino-C₂(CBN-C₂), cannabiorcol (CBN-C₁), cannabinodiol (CBND), cannabinodiolic acid (CBNDA), cannabinodivarin (CBDV), cannabitriol (CBT), 10-ethoxy-9-hydroxy-Δ^{6a}-tetrahydrocannabinol, 8,9-dihydroxy-Δ^{6a(10a)}-tetrahydrocannabinol (8,9-Di-OH-CBT-C₅), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ^{6a(10a)}-tetrahydrocannabinol (MEC), Δ⁹-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, and dodeca-2E,4E-dienoic acid isobutylamide.

In some embodiments, the target molecule is CBDA. In some embodiments, the target molecule is CBD. In some embodiments, the target molecule is THCA. In some embodiments, the target molecule is THC. In some embodiments, the target cannabinoid is selected from the group consisting of CBG, CBN, CBC, and THCV.

### Molecularly Imprinted Polymer:

In one aspect, the present disclosure is directed to methods for preparing molecularly imprinted polymer (“MIP”) absorbents or materials, MIP absorbents or materials prepared by such processes, and processes utilizing the MIP absorbents or materials of the present disclosure. The MIP absorbents and materials of the present disclosure are suitable for separating, extracting, or sequestering a specific cannabinoid, such as tetrahydrocannabinol (THC) and cannabidiol (CBD), from a mixture. In some embodiments, the MIP absorbents and materials of the present disclosure are suitable for separating, extracting, or sequestering multiple cannabinoids from a mixture (such as Cannabis or hemp extract).

Absorption-based processes are often designed to separate, extract, or sequester a specific molecular species or “target” molecule (e.g. target cannabinoid) from a mixture, either to isolate the target molecule (e.g., because of its value), remove a specific species from a mixture (e.g., because of its toxicity or other hazardous properties), or to detect the target molecule (or molecules associated with the target molecule).

Molecularly imprinted polymers are highly selective absorbents with absorption sites specifically tailored to bind to a particular target molecule. Examples of known MIPs and methods of preparing and using MIPs include those disclosed in U.S. Pat. Nos. 7,067,702; 7,319,038; 7,476,316; 7,678,870; 8,058,208; 8,591,842, and 9,504,988, which are incorporated by reference herein in their entirety for all purposes. These MIPs are copolymers prepared by polymerizing a polymerizable ligand for the target molecule (i.e., a “ligand monomer”) in a polymer matrix composed of one or more non-crosslinking monomers (e.g., styrene or other monomers which do not form a complex with the target molecule), and one or more crosslinking agents. Conventionally, the “templated” absorption sites characteristic of MIPs are prepared by forming an appropriate complex of the ligand monomer with the target molecule, then polymerizing the resulting target molecule-ligand monomer complex in the presence of one or more non-crosslinking monomers and at least one cross-linking agent, under suitable polymerization conditions. The resulting polymer structure comprises a matrix of the polymerized non-crosslinking monomer(s) with dispersed binding sites or cavities (“complexing cavities”) containing the target molecule, still complexed to the (now polymerized) ligand monomer. Because the polymerization is carried out in the presence of the target molecule, the target molecule forms a “template” so that the size and shape of the complexing cavity is specific to the particular target molecule, resulting in highly selective binding to the target molecule relative to other molecules.

Applicants have found that the selectivity advantages of conventional MIPs can be retained without the need to use the target molecule itself as a template for the binding site, by substituting an appropriately selected “surrogate” molecule for the target molecule. As will be exemplified herein, a MIP selective for target molecule “A” can be prepared by polymerizing a complex of a suitable surrogate molecule “B” with non-crosslinking monomer(s) and crosslinking monomer(s), provided that “A” and “B” complex to the monomers using the same physicochemical mechanism (i.e., functional properties), have similar size and/or shape (i.e., steric properties), and “B” is one or more of less expensive, less hazardous (i.e., toxic, radioactive), or more compatible with the polymerization conditions compared to “A.” The resulting “surrogate” templated MIPs, while perhaps somewhat less selective for the target molecule than those prepared using the conventional process (in which the target molecule serves as the molecular template) are much less expensive, safer to prepare, easier to manufacture and scale-up, etc., yet sufficiently selective in e.g., separation or extraction applications to be similar in performance to conventional MIPs, yet substantially lower in cost. Moreover, the “surrogate” templated MIPs of the present disclosure provide substantial improvements in overall separation process costs due to their combination of high performance at relatively low cost.

Various different physicochemical interactions between the monomers and target molecule can be exploited to prepare MIPs materials according to the disclosure including covalent, ionic, ion-dipole, hydrogen bonding, dipole-dipole, induced dipole or instantaneous dipole-induced dipole (i.e., London dispersion) attractive interactions, and minimizing coulombic and steric repulsive interactions. Thus, a cannabinoid surrogate having substantially the same functional properties as a target cannabinoid, would have, for example, covalent, ionic, ion-dipole, hydrogen bonding, dipole-dipole, induced dipole or instantaneous dipole-induced dipole (i.e., London dispersion) attractive interactions that are substantially the same as the target cannabinoid. When the target molecule is neutral (i.e., has no formal charge, such as the cannabinoids described herein), suitable uncharged monomers include but are not limited to monomers including functional groups such as imines (as described herein), amines, phosphines, esters, ethers, cryptands, thio ethers, Schiff bases and the like. Prior to polymerization with one or more non-crosslinking monomers and one or more cross-linking monomers to form the MIP bead, these monomers are mixed with the cannabinoid surrogate (or in some embodiments, target cannabinoid) which allows the monomers to “selfassemble” or coordinate to the cannabinoid surrogate (or target cannabinoid) such that during polymerization the cannabinoid surrogate (or target cannabinoid) is incorporated into the polymerized MIP bead. In some embodiments, the surrogate molecule is removed from the bead before use by displacement with an appropriate alternative molecule.

In some embodiments, the cannabinoid surrogate is covalently bonded to a polymerizable group (such as an acrylate) to provide a cannabinoid surrogate-containing monomer. In such embodiments, the cannabinoid surrogate-containing monomer is mixed with one or more non-crosslinking monomers and one or more cross-linking monomers prior to polymerization to, upon polymerization, provide an “unactivated” MIP bead, wherein the cannabinoid surrogate is covalently linked to the MIP bead. In some embodiments, the cannabinoid surrogate is removed (or stripped) from the “unactivated” MP to provide an “activated” MIP having complexing cavities that selectively bind a target cannabinoid.

In one aspect, the present disclosure provides a plurality of macroreticular polymer beads comprising a copolymer having a plurality of complexing cavities that selectively bind a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid and
wherein the cannabinoid surrogate is removed to provide a complexing cavity that selectively binds a target cannabinoid.

As described herein, in conventional MIPs, a target molecule surrogate is removed from the polymer (or stripped from) to provide a MIP that is selective for the target molecule. Surprisingly, Applicants have found that the selectivity advantages of conventional MIPs are retained without the need to remove the surrogate monomer from the MIP, resulting in selective binding to the target cannabinoids(s) relative to other non-target molecules. In the present disclosure, such embodiments are sometimes referred as “unactivated” macroreticular polymer beads or “unactivated” MIP(s) (e.g., Examples 9-11).

The “unactivated” MIPs of the present disclosure comprise a polymer matrix with dispersed binding sites or cavities (“complexing cavities”) containing the surrogate monomer (now polymerized), covalently complexed in the polymer matrix. Without being bound by any theory, the cannabinoid surrogate monomer thus acts as a “template” to define a cavity or absorption site within the polymerized matrix that is specific to the target molecule (e.g., has a shape or size corresponding to the target molecule). In some embodiments, the surrogate monomer is removed from the “unactivated” MIP prior use to provide an “activated” MIP of the present disclosure.

Thus, in one aspect, the present disclosure provides a plurality of macroreticular polymer beads comprising a copolymer that selectively binds a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid.

In one aspect, the present disclosure provides a plurality of macroreticular polymer beads comprising a copolymer having a plurality of complexing cavities which selectively bind a target cannabinoid, wherein the copolymer is prepared from:
(a) a cannabinoid surrogate,
(b) a non-crosslinking monomer, and
(c) a crosslinking monomer:
wherein the cannabinoid surrogate has substantially the same steric and functional properties of the target cannabinoid and
wherein the cannabinoid surrogate is removed to provide a complexing cavity that selectively binds a target cannabinoid.

Suitable surrogates can be selected by first characterizing the size, shape, and relevant physicochemical characteristics of the target molecule. Candidate surrogate molecules of similar molecular shape and size, and similar physicochemical characteristics can then be identified by, for example, molecular modeling using commercially available molecular modeling programs such as ChemBioDraw® Ultra 14.0. For example, if the target molecule is neutral, the surrogate molecule would be selected to have a similar size, shape, and polarity as the target molecule. Advantageously, the surrogate should be relatively inexpensive, non-toxic, and not interfere with the polymerization (i.e., should not form a highly unstable complex with the ligand monomer, poison the polymerization catalyst, inhibit the initiator, react with other monomers or polymerization solvents, be insoluble in the polymerization solvent, etc.). The balancing of these various factors renders the selection of surrogates suitable for various target molecules and separation processes, unpredictable. In some embodiments, the surrogate is not catechin.

Substantially the same size and shape (or substantially the same steric properties) means that space filling models of the target molecule (e.g., a target cannabinoid) and the surrogate (e.g. a target cannabinoid surrogate) if superimposed on each other such that the overlap between the volumes defined by the space filling models is maximized (e.g. determined by means of commercial molecular modeling programs such as ChemBioDraw® Ultra 14.0) would differ by no more than about 50%, for example, no more than about 50%, no more than about 45%, no more than about 40%, no more than about 35%, no more than about 30%, no more than about 25%, no more than about 20%, no more than about 15%, the more than about 10%, or no more than about 5%, inclusive of all ranges and subranges therebetween.

Alternatively, a surrogate that is substantially the same size and shape as the target molecule can be functionally defined by the selectivity of the resulting MIP material for the target molecule (e.g., target cannabinoid). Since the complexing cavity of the inventive MIP materials is templated by a surrogate molecule rather than the target molecule, the selectivity for the MIP material for the surrogate material would be higher than for the target molecule. However, to the extent that the size and shape of the surrogate molecule would be substantially the same as the size and shape of the target molecule, the resulting MIP material would have a relatively high selectivity coefficient for the target molecule. Accordingly, higher selectivities for the target molecule would be indicative that the sizes and shapes of the target and surrogate molecules are substantially similar. In some embodiments the selectivity coefficient of the MIP materials of the present disclosure for the target molecule, templated with a surrogate molecule, are greater than about 10. In some embodiments, the selectivity coefficient of the MIP materials of the present disclosure are greater than: about 15, about 20, about 25, about 30, about 35, about 40, about 45, about 50, about 100, about 150, about 200, about 300, about 400, about 500, about 600, about 700, about 800, about 900, or about 1000, inclusive of all ranges therebetween.

In some embodiments, the cannabinoid surrogate is olivetol, dimethoxy olivetol, 1,3-bis(acryloyl)olivetol, 1,3-bis(methacryloyl)olivetol, 1-methacryloylolivetol, 1-acrlyloylolivetol, 1,3-bis(4-vinylbenzoyl)olivetol, 1-(4-vinylbenzoyl)olivetol, 1-(allyl)olivetol, 1,3-bis(allyl)olivetol, 2-methylolivetol, 2-phenylolivetol, 2-cyclohexylolivetol, 2-methyl-1,3-dimethoxy-4-pentylbenzene, 2-phenyl-1,3-dimethoxy-4-pentylbenzene, or 2-cyclohexyl-1,3-dimethoxy-4-pentylbenzene.

In some embodiments, the cannabinoid surrogate has the formula:

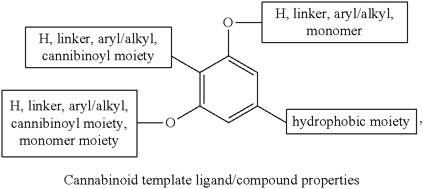

wherein the cannabinoid surrogate has substantially the same steric and functional properties of a target cannabinoid.

In some embodiments, the cannabinoid surrogate monomer has the formula:

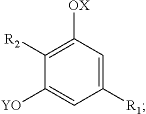

wherein:
R₁ is selected from the group consisting of H, alkyl, substituted alkyl, oxo substituted alkyl, acyl, substituted acyl, aryl, and substituted aryl,
X is selected from the group consisting of H, alkyl, substituted alkyl, oxosubstituted alkyl, acyl, substituted acyl, aryl, substituted aryl, polyether, alkenyl, benzoyl, substituted benzoyl, 4-vinylbenzoyl, 2-vinylbenzoyl, 3-vinylbenzoyl, allyl, methacryloyl, acryloyl, substituted acryloyl, carbamoyl, substituted carbamoyl, glycidyl methacrylate, and glycidyl acrylate;
Y is selected from the group consisting of H, cannabinoid group (linked or unlinked to R₂), alkyl, substituted alkyl, aryl, substituted aryl, polyether, alkenyl, benzoyl, substituted benzoyl, 4-vinylbenzoyl, 2-vinylbenzoyl, 3-vinylbenzoyl, allyl, methacryloyl, substituted methacryloyl, acryloyl, substituted acryloyl, carbamoyl, substituted carbamoyl, 2-hydroxyethyl methacrylate, 2-hydroxyethyl acrylate, glycidyl methacrylate, and glycidyl acrylate; and
R₂ is selected from the group consisting of H, cannabinoid group (linked or unlinked to Y), alkyl, substituted alkyl, aryl, substituted aryl, alkenyl, acyl, substituted acyl, alkyl substituted acyl, and aryl substituted acyl,
wherein the cannabinoid surrogate has substantially the same steric and functional properties of a target cannabinoid.

As used herein, the term, “cannabinoidyl group” means a substructure that has substantially the same steric and functional properties of a target cannabinoid. The cannabinoidyl group is selected from the following substructures:

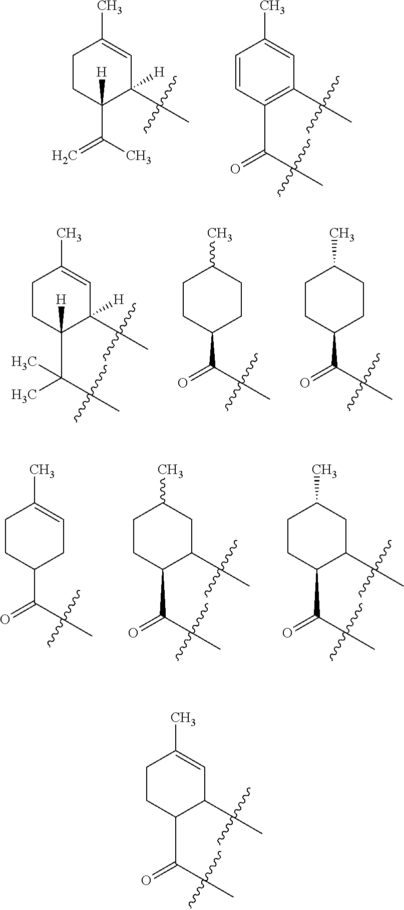

In some embodiments, the cannabinoid surrogate-containing monomer is a cannabinoid surrogate covalently bonded to one or more groups that may be polymerized (such as an acrylate).

In some embodiments, the cannabinoid surrogate-containing monomer has the formula:

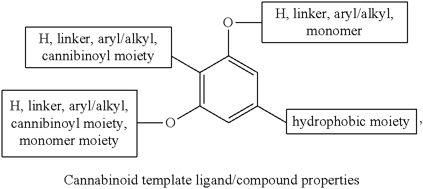

wherein at least one of the positions one or more groups that may be polymerized (such as an acrylate).

In some embodiments, the cannabinoid surrogate-containing monomer has the formula:

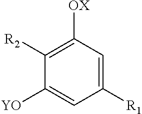

R₁ is selected from the group consisting of H, alkyl, substituted alkyl, oxosubstituted alkyl, acyl, substituted acyl, aryl, and substituted aryl,
X is selected from the group consisting of H, alkyl, substituted alkyl, oxosubstituted alkyl, acyl, substituted acyl, aryl, substituted aryl, polyether, alkenyl, benzoyl, substituted benzoyl, 4-vinylbenzoyl, 2-vinylbenzoyl, 3-vinylbenzoyl, allyl, methacryloyl, acryloyl, substituted acryloyl, carbamoyl, substituted carbamoyl, glycidyl methacrylate, and glycidyl acrylate;
Y is selected from the group consisting of H, cannabinoidyl group (linked or unlinked to R₂), alkyl, substituted alkyl, aryl, substituted aryl, polyether, alkenyl, benzoyl, substituted benzoyl, 4-vinylbenzoyl, 2-vinylbenzoyl, 3-vinylbenzoyl, allyl, methacryloyl, substituted methacryloyl, acryloyl, substituted acryloyl, carbamoyl, substituted carbamoyl, 2-hydroxyethyl methacrylate, 2-hydroxyethyl acrylate, glycidyl methacrylate, and glycidyl acrylate; and
R₂ is selected from the group consisting of H, cannabinoidyl group (linked or unlinked to Y), alkyl, substituted alkyl, aryl, substituted aryl, alkenyl, acyl, substituted acyl, alkyl substituted acyl, and aryl substituted acyl,
wherein at least one of the positions R₁, R₂, X and Y is substituted with one or more groups that may be polymerized (such as an acrylate).

In some embodiments, the cannabinoid surrogate-containing monomer has the formula:

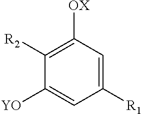

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, benzoyl, 4-vinylbenzoyl, 2-vinylbenzoyl, 3-vinylbenzoyl, allyl, methacryloyl, acryloyl, carbamoyl, glycidyl methacrylate, and glycidyl acrylate;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group;
wherein the cannabinoid surrogate has substantially the same steric and functional properties of a target cannabinoid and
wherein at least one of X and Y contains a polymerizable group. In some embodiments, the polymerizable group is a thermally polymerizable group. In some embodiments, the polymerizable group is a photopolymerizable group. In some embodiments, the polymerizable group is a free radical polymerizable group. In some embodiments, free radical polymerization may use any UV or thermal free radical initiator known to those skilled in the art, including free radical initiators disclosed herein.

In some embodiments, the cannabinoid surrogate is selected from the group consisting of:

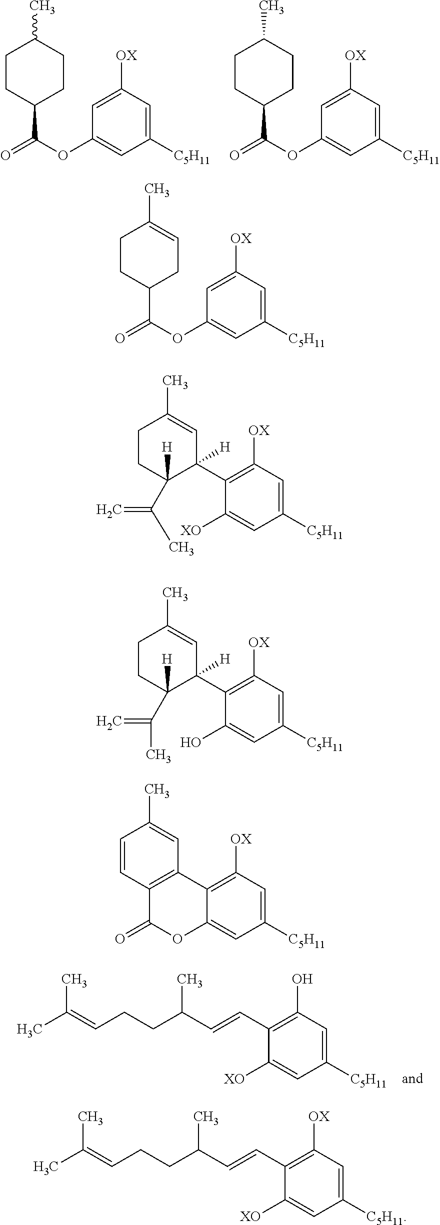

wherein X is a polymerizable group. In some embodiments, the polymerizable group is a thermally polymerizable group. In some embodiments, the polymerizable group is a photopolymerizable group. In some embodiments, the polymerizable group is a free radical polymerizable group. In some embodiments, free radical polymerization may use any UV or thermal free radical initiator known to those skilled in the art, including free radical initiators disclosed herein. In some embodiments, the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.

In some embodiments, of the cannabinoid surrogate-containing monomer, R₂ is the cannabinoidyl group and has the formula:

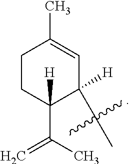

In some embodiments, the cannabinoid surrogate-containing monomer has the formula:

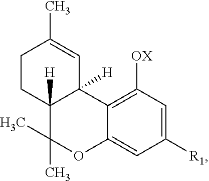

wherein
X contains a polymerizable group.

In some embodiments of X, the polymerizable group is a thermally polymerizable group. In some embodiments, the polymerizable group is a photopolymerizable group. In some embodiments, the polymerizable group is a free radical polymerizable group. In some embodiments, free radical polymerization may use any UV or thermal free radical initiator known to those skilled in the art, including free radical initiators disclosed herein.

In some embodiments of the cannabinoid surrogate-containing monomer wherein the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.

In some embodiments, the cannabinoid surrogate-containing monomer is selected from the group consisting of:

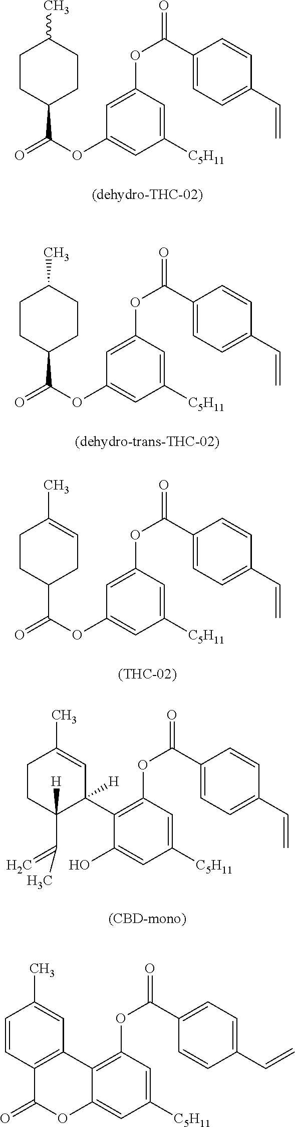

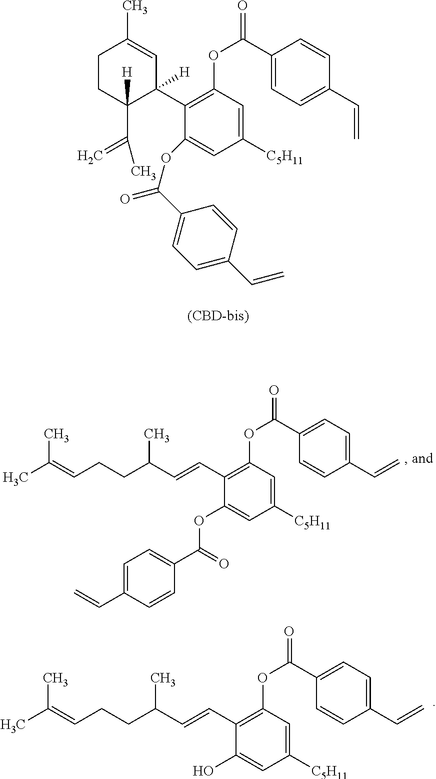

In some embodiments, the cannabinoid surrogate-containing monomer has the has the formula: the cannabinoid surrogate has the formula:

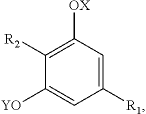

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, and benzoyl;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid.

In some embodiments, the cannabinoid surrogate is selected from the group consisting of:

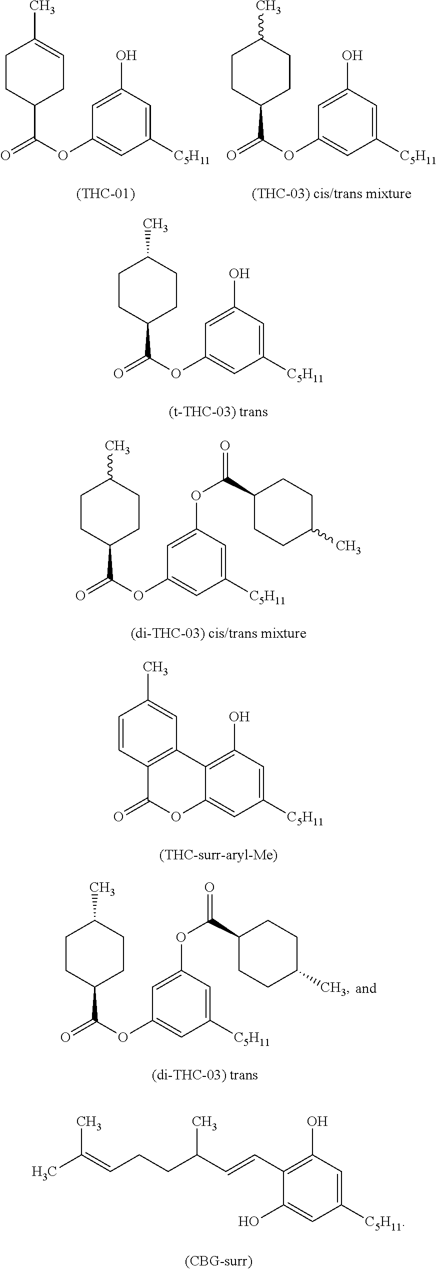

MIP beads according to the present disclosure can have any suitable shape, ranging from approximately spherical, to elongated, irregular (e.g., similar to the irregular shape of cottage cheese curds), or formed to specific desired shapes.

In various embodiments, it is desirable that the molecularly imprinted polymer be in the form of beads, particularly porous beads that have sufficient porosity so as to allow facile mass transport in and out of the bead.

The term “bead” refers to a plurality of particles with an average particle size ranging from about 250 μm to about 1.5 mm. In some embodiments, the average particle size of the beads can be about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, or about 1500 μm, including any ranges between any of these values. In some embodiments, the average particle size range is from about 0.3 mm to 1.1 mm.

In some embodiments, the MIP beads of the present disclosure have a substantially unimodal particle size distribution. In some embodiments, it is desirable for the MIP beads to have a bimodal or other multimodal particle size distribution.

In many processes, material handling or mass flow requirements dictate that the percentage of fine particles be low. Accordingly, in some embodiments, less that about 10% of the MIP beads of the present disclosure have a particle size less than about 250 μm. In some embodiments, less than about 5% or less than about 1% of the beads have a particle size less than about 250 μm. The average particle size of the beads may be measured by various analytical methods generally known in the art including, for example, ASTM D 1921-06.

In most embodiments, it is desirable that the beads of the present disclosure be porous to facilitate mass flow in and out of the bead. In some embodiments, the MIP beads of the present disclosure are characterized as “macroreticular” or “macroporous,” which refers to the presence of a network of pores having average pore diameters of greater than 100 nm. In various embodiments, polymer beads with average pore diameters ranging from 100 nm to 2.4 μm are prepared.

In some embodiments the average pore diameters can be about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, or about 2400 nm, including ranges between any of these values.

The beads can also be mesoporous, or include mesopores (in addition to macropores). The term “mesoporous” refers to porous networks having an average pore diameter from 10 nm to 100 nm. In some embodiments mesopore average pore diameters can be about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, or about 100 nm, including any ranges between any of these values.

In addition, the beads can also be microporous, or include micropores in addition to macropores and/or mesopores. The term “microporous” refers to porous networks having an average pore diameter less than 10 nm. In some embodiments micropore average pore diameters can be about 0.5 nm, about 1 nm, about 1.5 nm, about 2 nm, about 2.5 nm, about 3 nm, about 3.5 nm, about 4 nm, about 4.5 nm, or about 5 nm, or about 5.5 nm, about 6 nm, about 6.5 nm, about 7 nm, about 7.5 nm, about 8 nm, about 8.5 nm, about 9 nm, about 9.5 nm, or about 10 nm, including ranges between any of these values.

The macroreticular polymer beads have a surface area of about 0.1 to about 500 m²/g, for example about 0.1, about 0.5, about 1, about 5, about 10, about 15, about 20, about 30, about 40, about 50, about 60, about 70, about 80, about 90, about 100, about 150, about 200, about 250, about 300, about 350, about 400, about 450, or about 500 m²/g, inclusive of all ranges and subranges therebetween.

The structure and porosity of the beads are determined principally by the conditions of polymerization. The desired porosity of the bead can be achieved by the choice of surrogate/ligand monomer complex, non-crosslinking monomer and crosslinking agents and their amounts, as well as the composition of the reaction solvent(s) and optional pore forming additives or thixotropic agents. Porosity determines the size of the species, molecule or ion that may enter a specific structure and its rate of diffusion and exchange, as well as the rate of mass flow in and out of the bead structure.

The pore forming agents can significantly improve control of bead formation and can substantially improve the chemical and/or physical properties of the beads. In some embodiments, the pore forming agents may significantly improve the absorption or binding capacity of the MIP. In some embodiments, the pore forming agent is a poly(ethylene glycol) oligomer. Suitable poly(ethylene glycol) oligomers include PEG oligomers having an average molecular weight of about MW 400 to about MW 6000, including all values in between. In some embodiments, the pore forming agent is PEG, MW ˜1100. In some embodiment, the pore forming agent is PEG, MW ˜5800.

The thixotropic agents can significantly improve control of bead formation and substantially uniform bead or particle size. Suitable thixotropic agents employed herein are dependent on the type and amount of monomer employed and the suspending medium. The thixotropic agents can also advantageously act as suspension agents during the suspension polymerization process. Representative examples of such thixotropic agents include, but are not limited to, cellulose ethers such hydroxyethylcellulose, (commercially available under the trade name of “CELLOSIZE”), cross-linked polyacrylic acid such as those known under the name of “CARBOPOL” polyvinyl alcohols such as those known under the trade name of “RHODOVIOL”, boric acid, gums such as xanthan gum and the like and mixtures thereof, The amount of thixotropic agents can influence the size of the resin (i.e., the use of larger amounts of thixotropic agents often results in the formation of smaller resin particles).

The amount of the thixotropic agent is generally from about 1.5 to about 5 weight percent, based on the weight of the monomers in the monomer mixture. In some embodiments, the amount of the thixotropic agent is from about 1.5 to about 2.5 weight percent, based on the weight of the monomer or monomers (combination of monomers) in the monomer mixture.

Cross-linking (also crosslinking) agents or cross-linking monomers that impart rigidity or structural integrity to the MIP are known to those skilled in the art, and include di-, tri- and tetrafunctional acrylates or methacrylates, divinylbenzene (DVB), alkylene glycol and polyalkylene glycol diacrylates and methacrylates, including ethylene glycol dimethacrylate (EGDMA) and ethylene glycol diacrylate, vinyl or allyl acrylates or methacrylates, divinylbenzene, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or Bismuth-acrylamide, including hexamethylene bisacrylamide lanthanide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1, 1-dimethy 1-2-isocyanatoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12, 17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride and the like.

In some embodiments of the macroreticular polymer beads of the present disclosure, the crosslinking monomer is selected from the group consisting of alkylene glycols and polyalkylene glycol diacrylates, polyalkylene glycol methacrylates, vinyl acrylates, vinyl methacrylates, allyl acrylates or allyl methacrylates, divinylbenzene, diallyldiglycol dicarbonate, diallyl maleate, diallyl fumarate, diallyl itaconate, vinyl esters, the dimethacrylates or diacrylates of bis-phenol A or ethoxylated bis-phenol A, methylene bisacrylamide, polymethylene bisacrylamide, bismuth-acrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1, 1-dimethy 1-2-isocyanatoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12, 17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; and divinyl tin dichloride. In some embodiments of the macroreticular polymer beads of the present disclosure, the crosslinking monomer is divinylbenzene.

The MIP must have sufficient rigidity so that the target molecule may be easily removed without affecting the integrity of the polymer. In such cases where the polymer matrix is insufficiently rigid, crosslinking or other hardening agents can be introduced. In imprinted MIPs, the cross-linker (cross-linking agent or monomer) fulfills three major functions: 1) the cross-linker is important in controlling the morphology of the polymer matrix, whether it is gel-type, macroporous or a microgel powder; 2) it serves to stabilize the imprinted binding site (complexing cavity); and 3) it imparts mechanical stability to the polymer matrix. In some embodiments, high cross-link ratios are generally desired in order to provide permanently porous materials with adequate mechanical stability.

A wide variety of monomers may be used as a non-crosslinking monomer for synthesizing the MIP in accordance with the present disclosure. Suitable non-limiting examples of non-crosslinking monomers that can be used for preparing a MIP of the present disclosure include methylmethacrylate, other alkyl methacrylates, alkylacrylates, allyl or aryl acrylates and methacrylates, cyanoacrylate, styrene, substituted styrenes, methyl styrene (multisubstituted) including 1-methylstyrene; 3-methylstyrene; 4-methylstyrene, etc.; vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy) ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; aryloyl chloride; 1-α-acryloyloxy-β,β-dimethyl-γ-butyrolactone; N-acryloxy succinimide acryloxytris(hydroxymethyl)amino-methane; N-acryloyl chloride; N-acryloyl pyrrolidinone; N-acryloyl-tris(hydroxymethyl)amino methane; 2-aminoethyl methacrylate; N-(3-aminopropyl)methacrylamide; (o, m, or p)-amino-styrene; t-amyl methacrylate; 2-(1-aziridinyl)ethyl methacrylate; 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; -bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; t-butylacrylamide; butyl acrylate; butyl methacrylate; (o, m, p)-bromo styrene; t-butyl acrylate; 1-carvone; (S)-carvone; (−)-carvyl acetate; 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 1-(3-butenyl)-4-vinylbenzene; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethy 1-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); glycidyl acrylate, glycidyl methacrylate; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; (±)-linalool; linalyl acetate; p-mentha-1,8-diene; p-mentha-6,8-dien-2-ol; methyleneamino acetonitrile; methacrolein; [3-(methacryloylamino)-propyl] trimethylammonium chloride; methacrylamide; methacrylic acid; methacrylic anhydride; methacrylonitrile; methacryloyl chloride; 2-(methacryloyloxy)ethyl acetoacetate; (3-meth-acryloxypropyl)trimethoxy silane; 2-(methacryloxy)ethyl trimethylammonium methylsulfate; 2-methoxy propene (isopropenyl methyl ether); methyl-2-(bromomethyl)acrylate; 5-methyl-5-hexen-2-one; methyl methacrylate; N,N′methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; methyl vinyl ether; methyl-2-vinyloxirane; methyl vinyl sulfone; 4-methyl-5-vinylthiazole; myrcene; t-nitrostyrene; 3-nitrostyrene; 1-nonadecene; 1,8-nonadiene; 1-octadecene; 1, 7-octadiene; 7-40ctane-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyanoethylene; trans 3-chloroacrylic acid; 2-trifluoromethyl propene; 2-(trifluoromethyl)propenoic acid; 2,4,4′-trimethyl-1-pentene; 3, 5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethylsulfone; 4-(vinyl benzyl)-2-chloroethyl sulfone; N-(p-vinylbenzyl)-N,N′-dimethyl amine; 4-vinyl biphenyl (4-phenylstyrene); vinyl bromide; 2-vinyl butane; vinyl butyl ether; 9-vinyl carbazole; vinyl carbinol; vinyl cetyl ether; vinyl chloroacetate; vinyl hloroformate; vinyl crotanoate; vinyl peroxcyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinylquinoline; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinylquinoline; 1-vinyl silatrane; vinyl sulfone; vinyl sulfonic acid sodium salt; a-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy) silane; vinyl 2-valerate; vinyl benzoic acid; vinyl imidazole, vinylpyridine; vinylbenzylamine; Hydroxyethylmethacrylate (HEMA); and aminopropylmethacrylate.

In some embodiments, the non-crosslinking monomer is styrene. In some embodiments, the non-crosslinking monomer is styrene and the crosslinking monomer is divinylbenzene.

Acrylate-terminated or otherwise unsaturated urethanes, carbonates, and epoxies can also be used in the MIP. An example of an unsaturated carbonate is allyl diglycol carbonate. Unsaturated epoxies include, but are not limited to, glycidyl acrylate, glycidyl methacrylate, allyl glycidyl ether, and 1,2-epoxy-3-allyl propane.

The beads of the present disclosure can be prepared by various polymerization techniques. A polymer matrix can then be formed via a suitable polymerization technique in the presence of the surrogate/ligand monomer complex to form an imprinted resin. The resin product can be then be recovered. Non-limiting examples of suitable polymerization techniques can include aqueous suspension polymerization, inverse suspension polymerization (e.g. in perfluorocarbon), non-aqueous dispersion polymerization, two-stage swelling polymerization, aerosol polymerization, latex seeded emulsion polymerization, electropolymerization, and bulk polymerization on porous bead substrates. In some embodiments, the polymerization method is the aqueous suspension polymerization of a copolymerizable mixture of an organic phase containing non-crosslinking monomer, an optional crosslinker, and the surrogate/ligand monomer complex, and an aqueous phase containing at least one or more thixotropic agents.

In some embodiments of the present disclosure, a MIP is prepared by suspension polymerization of a surrogate/ligand monomer complex and other monomers as described herein. In the suspension polymerization procedure, the various phases can be thoroughly mixed separately prior to the start of the reaction and then added to the polymerization reaction vessel. While this mixing of the ingredients can be done in a vessel other than the reaction vessel, the mixing can alternatively be conducted in the polymerization reaction vessel under an inert atmosphere, particularly where the monomers being employed are subjected to oxidation. Further, in order to improve yields and selectivity of the final resin product, it is desirable that the ligand monomer be hydrolytically stable under polymerization conditions and in the final product. For example, the ligand monomer can be hydrolytically stable in a suspension polymerization formulation and under a water treatment environment such that hydrolysis is substantially avoided during polymerization and the useful life of the resin.

The polymerizable ligand/surrogate complex of the present disclosure can be polymerized under suspension polymerization conditions where the aqueous phase contains thixotropic agents such as polyvinyl alcohol and boric acid in water, and the organic phase comprises, for example, the polymerizable ligand/surrogate complex, styrene (non-crosslinking monomer), divinylbenzene (cross-linking monomer), organic solvents, and AIBN (initiator). The biphasic mixture is agitated, for example with a stirrer. By varying the temperature, agitation, polymerizable ligand/surrogate loading, solvent ratios, and degree of cross-linking, different beads structures and properties can be obtained. For example, spherical and porous beads of the desired size can be obtained by controlling the agitation or stirring during the polymerization. When the polymerization mixture is agitated to disperse the monomers dissolved in the organic reaction medium as droplets within the aqueous phase, suitably the droplets are of such size that when transformed into polymer beads, they are substantially spherical and porous, and of the desired size. Unsuitable reaction conditions can lead to the formation of no or very small beads, high surrogate losses to the aqueous phase, low overall yield, and insufficient porosity such that there is poor mass transfer to the complexing cavity.

Polymerization can be carried out at any suitable temperature. In some embodiments, the reaction is carried out at an elevated temperature, for example above about 50° C. in the presence of an optional initiator. Suitable initiators that can be used include but are not limited to benzoyl peroxide, diacetylperoxide, and azo-bisisobutyronitrile (AIBN). The amount of initiator employed can be within the range of about 0.005 to about 1.00% by weight, based on the weight of the monomer being polymerized. In the presence of an initiator, the temperature of reaction is maintained above that at which the initiator becomes active. Lower temperatures, e.g. about −30° C. to about 200° C., can be employed if high energy radiation is applied to initiate polymerization. Styrenic polymerizations can be thermally initiated.

Proper and sufficient agitation or stirring throughout the polymerization typically provides substantially spherical and porous beads having the desired size. For example, the polymerization mixture can be agitated to disperse the monomers (dissolved in the solvent organic phase) in the aqueous solvent phase by shear action, thereby forming droplets. By selecting the proper level of agitation, the droplets can be of such size that when transformed into polymer beads, they are substantially spherical and porous, and will have the desired size as discussed herein.

Various means are available to maintain the proper agitation. When polymerization is conducted in a reactor made of stainless steel, such a reactor can be fitted with a rotatable shaft having one or more agitator blades. When a round-bottom flask is used as a reactor, an overhead stirrer can be used to agitate the reaction medium. The amount of agitation necessary to obtain the desired results will vary depending upon the particular monomers being polymerized, as well as the particular polymer bead size desired. Therefore, the agitation speed such as the rpm (revolutions per minute) may be regulated within certain limits. Polymerization times can vary from about 3 hours to about 72 hours, depending on the reactivity of the monomers.

When polymerization is complete, the surrogate can be removed from the typically cross-linked polymer beads without substantially affecting the complexing cavity. Removal of the surrogate molecule provides e.g. a bead having a porous structure with complementary molecular cavities therein that has high binding affinity for the target molecule. In some embodiments, the surrogate is removed from the typically cross-linked polymer beads by hydrolysis.

Any suitable conditions effective to polymerize the monomers of the present disclosure to produce an MIP without dissociating the ligand/surrogate complex may be used. The monomers of the present disclosure may be polymerized by free radical polymerization, and the like. Any UV or thermal free radical initiator known to those skilled in the art can be used in the preferred free radical polymerization. Examples of UV and thermal initiators include benzoyl peroxide, acetyl peroxide, lauryl peroxide, azobisisobutyronitrile (AIBN), t-butyl peracetate, cumyl peroxide, t-butyl peroxide; t-butyl hydroperoxide, bis(isopropyl) peroxy-dicarbonate, benzoin methyl ether, 2,2′-azobis(2,4-dimethyl-valeronitrile), tertiary butyl peroctoate, phthalic peroxide, diethoxyacetophenone, t-butyl peroxypivalate, diethoxyacetophenone, 1-hydroxycyclohexyl phenyl ketone, 2,2-dimethyoxy-2-phenylacetophenone, and phenothiazine, diisopropylxanthogen disulfide, 2,2′-azobis-(2-amidinopropane); 2,2′-azobisisobutyronitrile-; 4,4′-azobis-(4-cyanovaleric acid); 1,1′-azobis-(cyclohexanecarbonitrile)-; 2,2′-azobis-(2,4-dimethyl valeronitrile); and the like and mixtures thereof.

The choice of monomer and cross-linking agent will be dictated by the chemical (hydrophilicity, chemical stability, degree of cross-linking, ability to graft to other surfaces, interactions with other molecules, etc.) and physical (porosity, morphology, mechanical stability, etc.) properties desired for the polymer. The amounts of ligand monomer/surrogate complex, monomer and crosslinking agents should be chosen to provide a crosslinked polymer exhibiting the desired structural integrity, porosity and hydrophilicity. The amounts can vary broadly, depending on the specific nature/reactivities of the ligand/surrogate complex, monomer and crosslinking agent chosen as well as the specific application and environment in which the polymer will ultimately be employed. The relative amounts of each reactant can be varied to achieve desired concentrations of ligand/surrogate complexes in the polymer support structure. Typically, the amount of ligand surrogate complex will be on the order of about 0.01 mmol to about 100 mmol percent of monomer, including: about 0.02, 0.05, 0.1, 0.2, 0.3, 0.5, 1, 2, 3, 4, 5, 10, 15, 20, 25, 30, 35, 40, 45, 50, 55, 60, 65, 70, 75, 80, 85, 90, 95, or 100 mmol percent of monomer. The amount of cross-linker is typically on the order of about 1.0 to about 10 mole percent, including about 1.5, 2, 3, 4, 5, 6, 7, 8, or 9 mole percent of monomer. The amount of a free radical initiator can be about 0.005 to 1 mole percent, including about 0.01, 0.05, 0.1, 0.5, 0.6, 0.7, 0.8, or 0.9 mole percent of monomer. (Molar percentages refer to the percentage relative to the total amount of monomers prior to polymerization.)

As such the ligand as described herein comprises all or nearly all of the monomer used in preparing the MIP with little to no supporting polymer backbone and crosslinking. Such a ligand monomer must be functionalized and soluble in the conditions of suspension polymerization and must still result in a final polymerized form that maintains the polymer qualities suitable for commercial use (rigidity, selectivity, reuse capability, temperature and pH resistance). MIP materials of the present invention are stable (physically and chemically) in a pH range of about 0-13 (including about 0, 1, 2, 3, 4, 5, 6, 7, 8, 9, 10, 11, 12, and 13, inclusive of all ranges there between), a temperature range of about 0-100° C. (including about 0, 10, 20, 30, 40, 50, 60, 70, 80, 90, 100° C., inclusive of all ranges there between), have a mass attrition of less than about 20 wt. % (including less than about 20, 15, 10, 9, 8, 7, 6, 5, 4, 3, 2, 1, 0.5, or approximately 0 wt. %, inclusive of all ranges there between), stability to at least about 20 you cycles (including about 2, 3, 4, 5, 6, 7, 8, 9, 10, 11, 12, 13, 14, 15, 16, 17, 18, 19, or 20, inclusive of all ranges there between) and a selectivity coefficient (as described herein) for the desired target molecule of at least about 40 (including at least about 40, 45, 50, 55, 60, 65, 70, 75, 80, 85, 90, 95, or 100, inclusive of all ranges there between). The solvent, temperature, and means of polymerization can be varied in order to obtain polymeric materials of optimal physical or chemical features, for example, porosity, stability, and hydrophilicity. The solvent will also be chosen based on its ability to solubilize all the various components of the reaction mixture, and form a desirable polymer morphology.

The degree of crosslinking can range from about 1% to about 95%. In some embodiments, the degree of crosslinking is from about 5% to about 80%.

Any solvent which provides suitable solubility and is compatible with the desired reaction to the conditions to form the MIP materials of the present disclosure may be used. In some embodiments in which the MIP material is prepared by suspension polymerization conditions, the solvent can be a mixture of organic solvents. For example, the solvent can include long chain aliphatic alcohols such as pentanols, hexanols, heptanols, octanols, nonanols, decanols, undecanols, dodecanols, including saturated and unsaturated isomers thereof (e.g., methyl and ethyl pentanols, methyl and ethyl hexanols, methyl and ethyl, hepatanols, etc.), aliphatic hydrocarbons (e.g., butanes, pentanes, hexanes, heptanes, etc.), aromatic hydrocarbons (e.g., benzene, toluene, xylenes, etc.), and combinations thereof.

The resin thus obtained is in the form of porous beads. Porous beads can have an open cell structure such that the majority of open volumes within the bead are interconnected with one another and external openings on surfaces of the bead.

In one aspect, the present disclosure provides macroreticular molecularly imprinted polymer beads prepared according to any of the methods disclosed herein.

The present disclosure provides methods for preparation of MIPs. MIPs can be prepared by modification of known techniques including but not limited to those described in U.S. Pat. Nos. 4,406,792, 4,415,655, 4,532,232, 4,935,365, 4,960, 762, 5,015,576, 5,110,883, 5,208,155, 5,310,648, 5,321,102, 30 5,372,719, 5,786,428, 6,063,637, and 6,593,142, and U.S. application Ser. No. 15/176,158 the entire contents of each of which are incorporated herein by reference in their entireties for all purposes.

In one aspect, the present disclosure provides methods of making a plurality of macroreticular polymer beads comprising:
(1) polymerizing:
   a) a cannabinoid surrogate-containing monomer,
   b) a non-crosslinking monomer, and
   c) a crosslinking monomer; and
(2) removing the cannabinoid surrogate thereby forming macroreticular polymer beads having cavities that selectively bind the target cannabinoid,
wherein the cannabinoid surrogate has substantially the same steric and functional properties of the target cannabinoid.

In one aspect, the present disclosure provides methods of preparing macroreticular molecularly imprinted polymer beads comprising.
(1) polymerizing:
   (a) a cannabinoid surrogate-containing monomer,
   (b) a non-crosslinking monomer, and
   (c) a crosslinking monomer;
   wherein the cannabinoid surrogate has substantially the same steric and functional properties of the target cannabinoid and
(2) removing the cannabinoid surrogate to provide a complexing cavity that selectively binds a target cannabinoid.

In one aspect, the present disclosure provides methods of preparing macroreticular molecularly imprinted polymer that selectively binds a target cannabinoid, the method comprising:
(1) polymerizing:
   (a) a cannabinoid surrogate-containing monomer,
   (b) a non-crosslinking monomer, and
   (c) a crosslinking monomer;
wherein the cannabinoid surrogate has substantially the same steric and functional properties of the target cannabinoid.

In some embodiments of the methods of preparing macroreticular molecularly imprinted polymers the cannabinoid-containing surrogate monomer, non-crosslinking monomer, and crosslinking monomer are each independently selected from any cannabinoid surrogate, non-crosslinking monomer, and crosslinking monomer disclosed herein. In some embodiments, polymerizing occurs in the presence of a pore forming additive.

In one aspect, the present disclosure provides methods of preparing macroreticular molecularly imprinted polymer beads, the method comprising:
(1) polymerizing:
   (a) cannabinoid surrogate,
   (b) a non-crosslinking monomer, and
   (c) a crosslinking monomer:
   wherein the cannabinoid surrogate has substantially the same steric and functional properties of the target cannabinoid and
(2) removing the cannabinoid surrogate to provide a complexing cavity that selectively binds a target cannabinoid.

In some embodiments of the methods of preparing macroreticular molecularly imprinted polymer beads, the cannabinoid surrogate, non-crosslinking monomer, and crosslinking monomer are each independently selected from any cannabinoid surrogate, non-crosslinking monomer, and crosslinking monomer disclosed herein. In some embodiments, polymerizing occurs in the presence of a pore forming additive.

In some embodiments, the present disclosure provides a method of selectively sequestering one or more target cannabinoids from a solution of one or more target cannabinoids admixed with other Cannabis or hemp extract components, comprising first contacting the macroreticular polymer beads of the present disclosure with a stripping solution, whereby the target cannabinoid surrogates are removed from the macroreticular polymer beads, then contacting the stripped beads with the solution, thereby selectively sequestering the target cannabinoid in the macroreticular polymer beads. The sequestered target cannabinoid is then stripped from the beads with a solution capable of displacing the target cannabinoid, thereby regenerating the beads for reuse in sequestering target molecules.

In some embodiments, the present disclosure provides methods for selectively sequestering one or more target cannabinoids from a solution of one or more target cannabinoids admixed with other Cannabis or hemp extract components, the method comprising:
providing macroreticular polymer beads of the present disclosure then contacting the beads with the solution, thereby selectively sequestering the target cannabinoid in the macroreticular polymer beads.

In some embodiments, the macroreticular polymer beads are an activated form of the macroreticular polymer beads. In some embodiments, the Cannabis or hemp plant extract components are semi-processed.

In some embodiments, the present disclosure provides methods for removing at least two target cannabinoids from a solution containing at least two target cannabinoids admixed with other cannabinoids, the method comprising:
(1) providing an activated form of a macroreticular polymer beads of the present disclosure having an affinity for a first target cannabinoid;
(2) contacting the solution with the macroreticular polymer beads, whereby the first target cannabinoid is selectively complexed in the beads,
(3) recovering a solution that is substantially free of the first target cannabinoid; and
(4) providing an activated form of a macroreticular polymer beads of the present disclosure having an affinity for a second target cannabinoid in the recovered solution;
(5) contacting the solution of Step (3) with the macroreticular polymer beads, whereby the second target cannabinoid is selectively complexed in the beads, and
(6) recovering a solution that is substantially free of the first and second target cannabinoids.

In some embodiments, the present disclosure provides methods for removing at least two target cannabinoids from a solution containing at least two target cannabinoids admixed with other cannabinoids, the method comprising:
(1) providing an unactivated form of the macroreticular polymer beads of the present disclosure having an affinity for a first target cannabinoid;
(2) contacting the solution with the macroreticular polymer beads, whereby the first target cannabinoid is selectively complexed in the macroreticular molecularly imprinted polymer beads,
(3) recovering a solution that is substantially free of the first target cannabinoid; and
(4) providing an unactivated form of the macroreticular molecularly imprinted polymer beads of the present disclosure, having an affinity for a second target cannabinoid in the recovered solution;
(5) contacting the solution of Step (3) with the macroreticular polymer beads, whereby the second target cannabinoid is selectively complexed in the beads, and
(6) recovering a solution that is substantially free of the first and second target cannabinoids.

In some embodiments, the method of the present disclosure comprises repeatedly contacting a cannabinoid-containing solution with the macroreticular molecularly imprinted polymer beads of the present disclosure until substantially all the target cannabinoids are complexed in the beads. In some embodiments, the method is repeated 1-10 more times. In some embodiments, the target cannabinoids are independently selected for each occurrence from any cannabinoid disclosed herein. In some embodiments, the target cannabinoids are THC and CBD. In some embodiments, the target cannabinoids are substantially all the cannabinoids present in the solution (such as a hemp or Cannabis extract).

In some embodiments, the present disclosure provides methods of removing TIC from a cannabinoid-containing solution comprising THC and CBD, the method comprising:
(1) contacting the solution with the macroreticular polymer beads of the present disclosure, whereby the THC and CBD are complexed in the beads;
(2) selectively stripping CBD from the macroreticular polymer beads; and
(3) recovering a stripped solution comprising CBD that is substantially THC-free.

In some embodiments, the methods further comprising selectively stripping THC from the macroreticular polymer beads.

In some embodiments, the present disclosure provides methods of purifying a cannabinoid-containing solution, the method comprising:
(1) contacting the solution with the macroreticular polymer beads of the present disclosure, whereby the cannabinoids in the solution are complexed in the beads and
(2) stripping the cannabinoids from the macroreticular polymer beads to provide a purified cannabinoid-containing solution.

In some embodiments of the present disclosure, the solution of one or more target cannabinoids admixed with other cannabinoids a Cannabis or hemp extract. In some embodiments, the solution of two or more target cannabinoids admixed with other cannabinoids a Cannabis or hemp extract. In some embodiments, the Cannabis or hemp plant extract components are crude or semi-processed.

Cannabinoids can be extracted from starting plant materials according to methods known in the art. For example, suitable extraction methods include maceration, percolation, solvent extraction, steam distillation (providing an essential oil) or vaporization.

Solvent extraction of cannabinoids from starting plant materials may be carried out using essentially any solvent that dissolves cannabinoids/cannabinoid acids, such as for example C₁-C₅ alcohols (e.g. ethanol, methanol), C₃-C₁₂ alkanes (e.g. liquid propane, liquid butane, pentane, hexane, or heptane), Norflurane (HFA134a), HFA227, carbon dioxide, and ethanol/water mixture. When solvents such as those listed above are used, the resultant primary extract typically contains nonspecific lipid-soluble material or “ballast” e.g. waxes, wax esters and glycerides, unsaturated fatty acid residues, terpenes, carotenes, and flavonoids. The primary extract may be further purified for example by “winterization”, e.g., to −20° C. followed by filtration to remove waxy ballast.

As used herein, the term “winterizing” or “winterization” refers to the process by which plant lipids and waxes are removed from a Cannabis extract. Persons of skill in the art will know how to winterize an extract. Briefly, winterization may be conducted by dissolving the Cannabis extract in a polar solvent (for example, ethanol) at sub-zero temperatures. Winterization separates the waxes and lipids from the oil, forcing them to collect at the top of the mixture for easy filtration/collection. In some embodiments, winterization is conducted by mixing ethanol and hash oil into a container and placing it into a sub-zero freezer.

In some embodiments, a solution of one or more target cannabinoids admixed with other Cannabis extract components is obtained by i) extraction of the Cannabis plant material with liquid CO₂ and/or ethanol and ii) winterization or partial winterization of the crude extract. In embodiment where free cannabinoids from the Cannabis plant material are desired, the plant material is decarboxylated by heating the material to a defined temperature for a defined time sufficient to decarboxylate cannabinoid acids to free cannabinoids prior to extraction.

In some embodiments, the solution of one or more target cannabinoids is prepared according to a process comprising the following steps: i) optional decarboxylation of the plant material; ii) extraction with liquid CO₂; iii) precipitation with C₁-C₅ alcohol to reduce the proportion of non-target cannabinoid materials; iv) removal of the precipitate (preferably by filtration); v) optional treatment with activated charcoal; and vi) evaporation to remove C₁-C₅ alcohol and water, thereby producing the solution of one or more target cannabinoids. Extraction techniques for cannabinoids can be found in U.S. Pat. No. 7,700,368, which is hereby incorporated by reference in its entirety.

In some embodiments, the solution of one or more target cannabinoids is prepared according to a process comprising the following steps: i) optional CO₂ extraction from plant matter; ii) ethanol extraction for crude cannabinoids, plant waxes, and plant oils (crude extract); iii) winterization of the crude extract to remove the waxy ballast (e.g., at −80° C. for 24 hours); iv) filtration, and v) ethanol recovery and optional in-vessel decarboxylation of the winterized crude.

In some embodiments, the present disclosure provides methods for selectively sequestering one or more target cannabinoids from a solution of one or more target cannabinoids admixed with other cannabinoids, the method comprising: first contacting the macroreticular polymer beads of the present disclosure with a hydrolyzing solution, whereby the cannabinoid surrogates are removed from the macroreticular polymer beads, then contacting the stripped beads with the cannabinoid-containing solution, thereby selectively sequestering the target cannabinoid in the macroreticular polymer beads.

In some embodiments, the present disclosure provides methods of selectively sequestering one or more target cannabinoids from a solution of one or more target cannabinoids admixed with other cannabinoids, the method comprising: contacting the macroreticular molecularly imprinted polymer of the present disclosure with the cannabinoid-containing solution, thereby selectively sequestering the target cannabinoid in the macroreticular polymer beads.

In some embodiments, the methods for selectively sequestering one or more target cannabinoids from a solution of one or more target cannabinoids admixed with other cannabinoids, further comprises stripping the target cannabinoid from the macroreticular polymer beads.

In some embodiments, the present disclosure provides methods for removing THC from a cannabinoid-containing solution comprising THC and CBD, the method comprising:
(1) contacting the solution with the macroreticular polymer beads of the present disclosure, whereby the THC is selectively complexed in the beads, and
(2) recovering a solution comprising CBD that is substantially THC-free.

In some embodiments, the methods for removing THC from a cannabinoid-containing solution comprising THC and CBD, further comprise stripping the beads with a stripping solution, whereby the THC is substantially removed from the beads.

In some embodiments, the cannabinoid-containing solution is prepared by a process, comprising:
i) providing cannabinoid containing plant trim;
ii) extracting the plant trim with an extraction solvent; and
iii) optionally winterizing and/or filtering and/or distilling the extract to provide the solution.

In some embodiments, the solution is a solution of one or more target cannabinoids admixed with other cannabinoids. In some embodiments, the solution is a solution of at least two target cannabinoids admixed with other cannabinoids. In some embodiments, the solution is a Cannabis or hemp extract, such as, a Cannabis or hemp extract wherein the extract components are semi-processed.

In some embodiments, the extraction solvent is an alcohol/water mixture. In some embodiments, the alcohol is an optionally substituted C₁-C₆ alcohol. In some embodiments, the alcohol is an optionally substituted C₁-C₃ alcohol. In some embodiments, the alcohol is selected from the group consisting of methanol, ethanol and isopropanol.

In some embodiments, the stripping solution is a solution capable of displacing the target cannabinoid, thereby regenerating the beads for reuse in sequestering target cannabinoids. In some embodiments, the stripping solution is an alcohol/water mixture. In some embodiments, the stripping solution is ethanol or an ethanol/water mixture. In some embodiments, the stripping solution is 50-70% alcohol/water. In some embodiments, the stripping solution is 50-70% ethanol/water. In some embodiments, the stripping solution is an alcohol. In some embodiments, the alcohol is a C₁-C₅ alcohol. In some embodiments, the alcohol is a C₁-C₃ alcohol. In some embodiments, the alcohol is selected from the group consisting of methanol, ethanol and isopropanol.

In some embodiments, the stripping provides a substantially pure (or substantially purified) target cannabinoid(s) having a chromatographic purity of about or at least about 85%, 86%, 87%, 88%, 89%, 90%, 91%, 92%, 93%, 94%, 95%, 96%, 97%, 98%, 99% or more, including ranges between any of these values.

In some embodiments, the stripping provides a substantially pure (or substantially purified) target cannabinoid having a purity of about or at least about 85% w/w, 86% w/w, 87% w/w, 88% w/w, 89% w/w, 90% w/w, 91% w/w, 92% w/w, 93% w/w, 94% w/w, 95% w/w, 96% w/w, 97% w/w, 98% w/w, 99% w/w or more, including ranges between any of these values. In some embodiments, stripping provides substantially pure target cannabinoid. In some embodiments, the target cannabinoid is CBD and stripping provides a substantially pure CBD comprising <1% w/w THC. In some embodiments, the stripping provides a substantially pure CBD comprising less than, or less than or equal to about: 0.9% w/w, about 0.85% w/w, about 0.80% w/w, about 0.75% w/w, about 0.70% w/w, about 0.65% w/w, about 0.60% w/w, about 0.55% w/w, about 0.50% w/w, about 0.45% w/w, about 0.40% w/w, about 0.30% w/w, about 0.25% w/w, about 0.20% w/w, about 0.15% w/w, about 0.10% w/w, about 0.09% w/w, about 0.08% w/w, about 0.07% w/w, about 0.06% w/w, about 0.05% w/w, about 0.04% w/w, about 0.03% w/w, about 0.025% w/w, about 0.02% w/w, about 0.015% w/w or about 0.01% w/w; including all ranges there between. In some embodiments, the stripping provides a substantially pure CBD comprising ≤0.3% THC. In some embodiments, the stripping provides a substantially pure CBD comprising <0.3% w/w THC. In some embodiments, the stripping solution provides a substantially pure CBD comprising <0.09% THC. In some embodiments, the stripping solution provides a substantially pure CBD comprising ≤0.09% THC. In some embodiments, the stripping provides a substantially pure CBD comprising <0.01% w/w THC. In some embodiments, the stripping provides a substantially pure CBD comprising ≤0.01% w/w THC. In some embodiments, the CBD is essentially free of THC. In some embodiments, THC is not detectable in the substantially pure CBD.

In some embodiments, the substantially pure target cannabinoid(s) comprise: less than 5%, less than 2%, less than 1.5%, less than 1% or less 0.5% non-target cannabinoid(s), including ranges between any of these values. In some embodiments, purity refers to chromatographic purity. In some embodiments, purity refers to purity (w/w %). In some embodiments, the target and non-target cannabinoid(s) are different and independently selected from any cannabinoid disclosed herein.

In some embodiments, the methods of the present disclosure provide a solution enriched in target cannabinoid(s). Such preparations, for example, encompass solutions having at least 80%, at least 85%, at least 90%, or more, of the target cannabinoid(s) including ranges between any of these values.

In some embodiments, the purified cannabinoid-containing solution contains less than about 20 ppm of heavy metals. In some embodiments, the heavy metal impurities do not exceed about 10 ppm. In some embodiments the purified cannabinoid-containing solution comprises less than about 10 ppm, less than about 9.5 ppm, less than about 9 ppm, less than about 8.5 ppm, less than about 8 ppm, less than about 7.5 ppm, less than about 7.0 ppm, less than about 6.5 ppm, less than about 6.0 ppm, less than about 5.5 ppm, less than about 5.0 ppm, less than about 4.5 ppm, less than about 4.0 ppm, less than about 3.5 ppm, less than about 3.0 ppm, less than about 2.5 ppm, less than about 2.0 ppm, less than about 1.5 ppm, less than about 1.0 ppm, less than about 0.5 ppm, less than about 0.4 ppm, less than about 0.3 ppm, less that about 0.2 ppm, or less than about 0.1 ppm of heavy metal(s), including all ranges therebetween. In some embodiments, the heavy metal is independently selected from one or more heavy metal selected from the group consisting of arsenic, cadmium, chromium, lead, and mercury.

In some embodiments, the purified cannabinoid containing solution is substantially free of residual pesticides. In some embodiments the purified cannabinoid-containing solution comprises less than about 10 ppm, less than about 9.5 ppm, less than about 9 ppm, less than about 8.5 ppm, less than about 8 ppm, less than about 7.5 ppm, less than about 7.0 ppm, less than about 6.5 ppm, less than about 6.0 ppm, less than about 5.5 ppm, less than about 5.0 ppm, less than about 4.5 ppm, less than about 4.0 ppm, less than about 3.5 ppm, less than about 3.0 ppm, less than about 2.5 ppm, less than about 2.0 ppm, less than about 1.5 ppm, less than about 1.0 ppm, less than about 0.5 ppm, less than about 0.4 ppm, less than about 0.3 ppm, less that about 0.2 ppm, or less than about 0.1 ppm residual pesticides(s), including all ranges there between. In some embodiments, the purified cannabinoid-containing solution comprises less than about 0.1 ppm residual pesticide(s)

In some embodiments, the pesticide is selected from the group consisting of: Aldicarb, Carbofuran, Chlordane, Chlorfenapyr, Chlorpyrifos, Coumaphos, Daminozide, Dichlorvos, Dimethoate, Ethoprophos, Etofenoprox, Fenoxycarb, Fipronil, Imazalil, Methiocarb, Methyl Parathion, Mevinphos, Paclobutrazol, Propoxur, Spiroxamine, and Thiacloprid.

In some embodiments, the pesticide is selected from the group consisting of: Abamectin, Acephate, Acequinocyl, Acetamiprid, Aldicarb, Azoxystrobin, Bifenazate, Bifenthrin, Boscalid, Captan, Carbaryl, Chlorantraniliprole, Chlorfenapyr, Clofentezine, Cyfluthrin, Coumaphos, Cypermethrin, Daminozide, Diazinon, Dichlorvos (DDVP), Dimethoate, Dimethomorph, Ethoprop (hos), Etofenprox, Etoxazole, Fenhexamid, Fenoxycarb, Fenpyroximate, Fipronil, Flonicamid, Fludioxonil, Hexythiazox, Imazalil, Imadacloprid, Kresoxim-methyl, Malathion, Metalaxyl, Methiocarb, Methomyl, Methyl-parathion, Mevinphos, Myclobutanil, Naled, Oxamyl, Paclobutrazol, Pentachloronitrobenzene, Permethrin, Phosmet, Piperonyl butoxide, Prallethrin, Propiconazole (Tilt), Propoxur, Pyrethrins, Pyridaben, Spinetoram, Spinosad, Spiromesifen, Spirotetramat, Spiroxamine, Tebuconazole, Thiacloprid, Thiamethoxam, and Trifloxystrobin.

In some embodiments, the methods disclosed herein reduce the amount of “non-target”, i.e. non-cannabinoid, in the extract and/or provide a degree of separation/fractionation of the various cannabinoid/cannabinoid acid components of the crude plant extract. In some embodiments, the product of the methods disclosed herein is collected in multiple fractions, which may then be tested for the presence of the target cannabinoid using any suitable analytical technique (e.g. HPLC, TLC etc.). Fractions enriched in the target cannabinoid may then be selected. In some embodiments, the method may optionally include a further purification step. In some embodiments, the methods of the present disclosure provide substantially purified target cannabinoid(s). In some embodiments, the methods disclosed herein provide partially purified target cannabinoid(s).

Various MIP materials of the present disclosure can be reused (regenerated) more than once and frequently up to about 30 times or more, depending on the particular resin and the treated liquid medium. In some embodiments, regeneration can be accomplished in much the same manner as removal of the original cannabinoid surrogate, e.g. stripping or washing with an appropriate solution.

In some embodiments, the MIP materials are not regenerated.

Macroreticular MIP beads are particularly useful for selectively removing or adsorbing target dissolved species from solutions. In some embodiments, the solution is a crude or semi-processed hemp plant extract. In some embodiments, the solution is a crude or semi-processed Cannabis extract.

The MIP materials (e.g., beads or macroreticular beads) according to the present disclosure are selective for the target molecule(s) (e.g., CBD and/or THC). The selectivity of the MIP material to bind species “A” in a mixture of “A” and species “B” can be characterized by a “selectivity coefficient” using the following relationship:

$$Selectivity coefficient for A = \frac{[ {A}^{′} ] [ B ]}{[ A ] [ {B}^{′} ]}$$

where “[A]” and “[B]” refer to the molar concentration of A and B in solution, and “[A′]” and “[B′]” refer to the concentration of complexed “A” and “B” in the MIP material.

For most separations, the selectivity coefficient for the target cannabinoids(s) versus other species (e.g. non-cannabinoids or non-target cannabinoids) in the mixture to be separated should be at least about 1.1, at least about 1.2, at least about 1.3, at least about 1.4, at least about 1.5, at least about 2, at least about 5, at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 70, at least about 80, at least about 90, at least about 100, at least about 200, at least about 300, at least about 400, at least about 500, including ranges between any of these values.

As used herein, the term “bind,” “binding,” “bond,”, “bonded,” or “bonding” refers to the physical phenomenon of chemical species being held together by attraction of atoms to each other through sharing, as well as exchanging, of electrons or protons. This term includes bond types such as: ionic, coordinate, hydrogen bonds, covalent, polar covalent, or coordinate covalent. Other terms used for bonds such as banana bonds, aromatic bonds, or metallic bonds are also included within the meaning of this term. The selective binding interactions refer to preferential and reversible binding exhibited by the MIP for a target molecule (such as CBD and/or THC), as described herein.

In some embodiments, the present disclosure provides methods of making a plurality of macroreticular polymer beads comprising:
(1) polymerizing:
a) a functionalized monomer capable of post-polymerization modification;
b) a non-crosslinking monomer, and
c) a crosslinking monomer and
(2) reacting the polymer of Step (1) with a cannabinoid surrogate monomer thereby forming macroreticular polymer beads that selectively bind the target cannabinoid, wherein the cannabinoid surrogate monomer has steric and functional properties to selectively bind the target cannabinoid.

In some embodiments, the present disclosure provides a compound selected from the group consisting of:

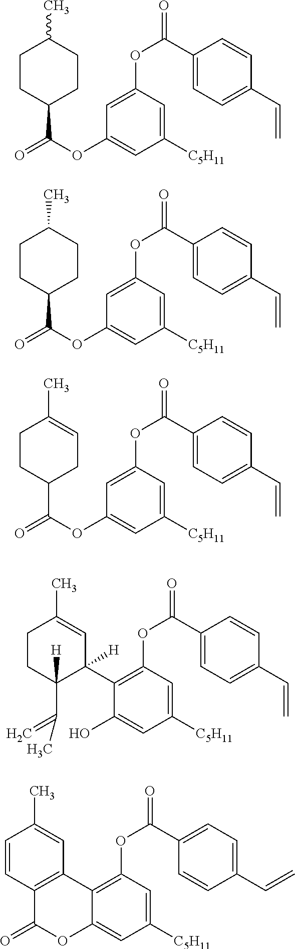

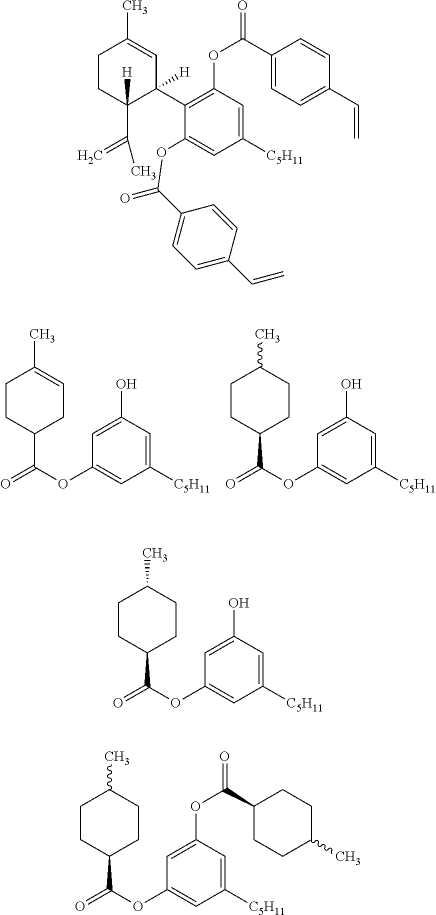

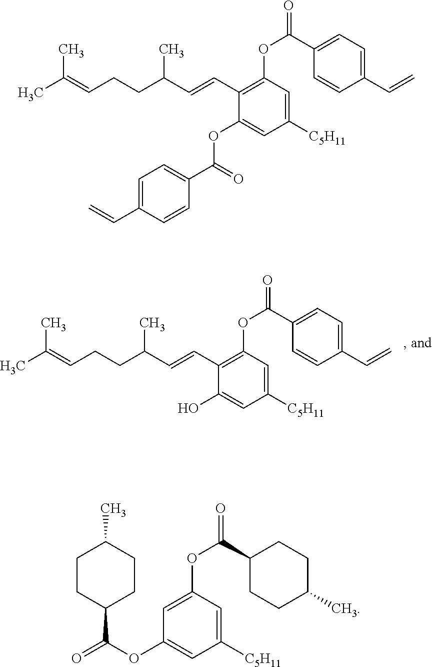

Throughout the description, where methods or processes are described as having, including, or comprising specific process steps, the processes also consist essentially of, or consist of, the recited processing steps. Further, it should be understood that the order of steps or order for performing certain actions is immaterial so long as the method remains operable. Moreover, two or more steps or actions can be conducted simultaneously.

### EXAMPLES

Reversed-phase high performance liquid chromatography (RP-HPLC) method for the detection of target analytes in loading/unloading solutions.

Instrument. An HP 1050 chromatography system equipped with an HP 1050 gradient pump, HP 1050 DAD UV/Vis detector, HP 1050 21-place autosampler, a Millipore column heater, Agilent Eclipse Plus column (C 18 silica, 3.5 μm particle size, 300 A pore size, 3.0×100 mm). Data collection is accomplished by Agilent ChemStation software which was further utilized for the analysis.

Conditions:

| | |
|---|---|
| Sample: | Stock solution prepared in water/ethanol 40/60, |
| | diluted 1/10 in Solvent A |
| Solvent A: | water/0.1% TFA |
| Solvent B: | MeCN/0.1% Formic acid |
| Flow rate: | 0.5 mL/min |
| Temperature: | 30° C. |
| UV: | 254/280 nm |
| Injection: | 0.100 mL |
| Gradient conditions: | 5% B for 2 min; 5-100% B over 6 min; |
| | 100% B for 12 min |

| Component | Peak (min) |
|---|---|
| Catechin (control) | 7.17 |
| CBD | 10.43 |
| THC-01 (template) | 10.93 |

### Example 1. Preparation of Cannabidiol Monomer—Mono-Functional (CBD-Mono)

To a 2000 mL jacketed round-bottom flask equipped with a magnetic stir bar was added 8.03 g CBD and 4.17 g 4-vinylbenzoic acid and 500 mL dichloromethane. The flask was cooled to 0° C. using a recirculating chiller bath, followed by the addition of 6.52 g of DCC and 0.203 g of DMAP. After the addition was complete, the mixture was allowed to warm to ambient temperature and stirred until complete by silica gel TLC (Rf 0.35 in 10% EtOAc/hexanes). At this time, the mixture was filtered, washed with 200 mL of 1 N aq. HCl, 200 mL of saturated brine solution, dried over Na₂SO₄, filtered and concentrated to yield crude product as an oil. Purification by silica gel chromatography (200 g silica gel, 0-2.5% EtOAc in hexanes) yielded 3.20 g pure product (28%) as a waxy solid.

¹H NMR (400 MHz, DMSO-d₆): δ 0.85 (m, 6H), 1.00-1.35 (m, 12H), 1.35-1.75 (m, 22H), 2.28 (t, 1H), 2.45 (t, 2H), 2.65 (bs, 1H), 3.01 (t, 1H), 3.81 (m, 1H), 4.45 (m, 4H), 5.05 (d, 2H), 5.45 (d, 1H), 6.02 (m, 3H), 6.36 (s, 1H), 6.50 (s, 1H), 6.83 (dd, 1H), 7.68 (d, 2H), 8.01 (d, 2H), 8.64 (bs, 1H), 9.40 (s, 1H).

### Example 2. Preparation of Cannabidiol Bis Monomer—Bis-Functional (CBD-Bis)

To a 500 mL round-bottom flask equipped with a magnetic stir bar was added 4.03 g CBD and 5.15 g 4-vinylbenzoic acid and 250 mL dichloromethane. The mixture was stirred at ambient temperature to dissolve the solids followed by the addition of 7.82 g of DCC and 0.148 g of DMAP. After the addition was complete, the mixture was at ambient temperature until complete by silica gel TLC (Rf 0.51 in 10% EtOAc/hexanes). At this time, the mixture was filtered, washed with 100 mL of 1 N aq. HCl, 100 mL of saturated brine solution, dried over Na₂SO₄, filtered and concentrated to yield crude product as an oil. Purification by silica gel chromatography (175 g silica gel, 0-2.% EtOAc in hexanes) yielded 4.12 g pure product (56%) as a white waxy solid.

¹H NMR (400 MHz, DMSO-d₆): δ 0.85 (m, 6H), 1.10 (m, 4H), 1.30 (m, 8H), 1.40-1.70 (m, 18H), 2.30 (t, 2H), 2.60 (t, 2H), 2.70 (t, 1H), 3.02 (t, 1H), 3.45 (d, 1H), 3.85 (d, 1H), 4.48 (dd, 2H), 5.07 (d, 2H), 5.47 (d, 2H), 6.01 (s, 2H), 6.06 (d, 2H), 6.85 (dd, 2H), 6.98 (s, 2H), 7.71 (d, 4H), 8.06 (d, 4H), 8.65 (bs, 1H).

### Example 3. Exemplary Synthesis of Macroreticular Beads with CBD-Mono (KOH Hydrolysis)

Preparation of Aqueous Phase

Polyvinyl alcohol (PVOH, average Mw 89,000-98,000, 99+% hydrolyzed, 5.13 g) is dissolved in water (270 mL) through gentle heating to 80° C. 2.21 g of boric acid is dissolved in 68 mL water and slowly added when the PVOH cools to 50° C.

Preparation of the Organic Phase and Polymerization

1.25 g of Pluronic P123 (PEG, MW ˜5800) is combined with 25.0 mL of ethylhexanol in a 125 mL Erlenmeyer flask equipped with a stir bar and allowed stir or sonicated until fully dissolved. 0.75 g of CBD-mono is added and allowed stir until fully dissolved. 19.9 mL of styrene and 6.9 mL of divinylbenzene are pre-mixed and combined with the solution of CBD-mono monomer, and allowed to stir, covered with a septum, under ambient conditions. 0.5 g of AIBN is added to the solution and dissolved completely. When dissolved, the solution is added to an addition funnel and degassed for 10 minutes. The aqueous is added to the reactor and degassed for 10 minutes while heating 80° C. When the temperature reaches 80° C., the solution is added to the aqueous phase at a rate of 1 mL/s. The reaction is allowed to proceed, with continuous agitation for approximately 8 hours.

Post-Reaction Bead Cleanup

Upon completion of the reaction, the reaction suspension is added to a 500 mL separatory funnel and allowed to settle. The aqueous phase is removed and the beads are further washed with 250 mL of deionized water. The beads are then collected by Buchner filtration (Whatman Grade 1 paper) and washed with water and methanol. The beads are placed in a Soxhlet extractor and extracted with acetone. The extracted beads are removed from the thimble and dried at 70° C. The beads are fractionated by size using the appropriate mesh sieve (#16 and #20/50 mesh beads are collected). The beads can be stored dry indefinitely at ambient temperature. CBD-mono beads were used directly for cannabinoid studies or were further activated by hydrolysis of the covalently bound CBD monomer.

Bead Activation (KOH Hydrolysis)

2 g of dried beads were added to a 100 mL round bottom flask followed by 50 mL of a 1:1 solution of 5% aqueous KOH and methanol. The suspension was heated to reflux for 12-18 hours (solution gains color during this time). The beads are allowed to cool and are filtered, and washed with water. The beads were then added to a 100 mL round bottom flask followed by 50 mL of a 1:1 solution of 0.5 M HCl and methanol. The suspension was heated to reflux for 12-18 hours. The beads are allowed to cool and are filtered, washed with water and then methanol. The activated beads are dried at 70° C. and stored at ambient temperature until use.

### Example 4. Exemplary Synthesis of Macroreticular Beads with CBD-Bis

Preparation of Aqueous Phase

Polyvinyl alcohol (PVOH, average Mw 89,000-98,000, 99+% hydrolyzed, 5.13 g) is dissolved in water (270 mL) through gentle heating to 80° C. 2.21 g of boric acid is dissolved in 68 mL water and slowly added when the PVOH cools to 50° C.

Preparation of the Organic Phase and Polymerization

1.25 g of Pluronic P123 (PEG, MW ˜5800) is combined with 25.0 mL of ethylhexanol in a 125 mL Erlenmeyer flask equipped with a stir bar and allowed stir or sonicated until fully dissolved. 0.75 g of CBD-bis is added and allowed stir until fully dissolved. 19.9 mL of styrene and 6.9 mL of divinylbenzene are combined with the solution of CBD-bis monomer, and allowed to stir, covered with a septum, under ambient conditions. 0.5 g of AIBN is added to the solution and dissolved completely. When dissolved, the solution is added to an addition funnel and degassed for 10 minutes. The aqueous is added to the reactor and degassed for 10 minutes while heating 80° C. When the temperature reaches 80° C., the solution is added to the aqueous phase at a rate of 1 mL/s. The reaction is allowed to proceed, with continuous agitation for approximately 8 hours.

Post-Reaction Bead Cleanup

Upon completion of the reaction, the reaction suspension is added to a 500 mL separatory funnel and allowed to settle. The aqueous phase is removed and the beads are further washed with 250 mL of deionized water. The beads are then collected by Buchner filtration (Whatman Grade 1 paper) and washed with water and methanol. The beads are placed in a Soxhlet extractor and extracted with acetone. The extracted beads are removed from the thimble and dried at 70° C. The beads are fractionated by size using the appropriate mesh sieve (#16 and #20/50 mesh beads are collected). The beads can be stored dry indefinitely at ambient temperature. CBD-bis beads were used directly for cannabinoid studies or were further activated by hydrolysis of the covalently bound CBD monomer.

Bead Activation (NaOMe Hydrolysis)

2 g of dried beads were added to a 100 mL round bottom flask followed by 50 mL of a 12.5 wt % NaOMe in methanol. The suspension was heated to reflux for 12-18 hours (solution gains color during this time). The beads are allowed to cool and are filtered, and washed with 1:1 water/methanol. The beads were then added to a 100 mL round bottom flask followed by 50 mL of a 1:1 solution of 0.5 M HCl and methanol. The suspension was heated to reflux for 12-18 hours. The beads are allowed to cool and are filtered, washed with water and then methanol. The activated beads are dried at 70° C. and stored at ambient temperature until use.

### Example 5. Exemplary Synthesis of Macroreticular Beads with CBD-Mono (Pendant Ligand)

Preparation of Aqueous Phase

Polyvinyl alcohol (PVOH, average Mw 89,000-98,000, 99+% hydrolyzed, 5.13 g) is dissolved in water (270 mL) through gentle heating to 80° C. 2.21 g of boric acid is dissolved in 68 mL water and slowly added when the PVOH cools to 50° C.

Preparation of the Organic Phase and Polymerization

1.25 g of Pluronic P123 (PEG, MW ˜5800) is combined with 25.0 mL of ethylhexanol in a 125 mL Erlenmeyer flask equipped with a stir bar and allowed stir or sonicated until fully dissolved. 0.75 g of CBD-mono is added and allowed stir until fully dissolved. 19.9 mL of styrene and 6.9 mL of divinylbenzene are pre-mixed and combined with the solution of CBD-mono monomer, and allowed to stir, covered with a septum, under ambient conditions. 0.5 g of AIBN is added to the solution and dissolved completely. When dissolved, the solution is added to an addition funnel and degassed for 10 minutes. The aqueous is added to the reactor and degassed for 10 minutes while heating 80° C. When the temperature reaches 80° C., the solution is added to the aqueous phase at a rate of 1 mL/s. The reaction is allowed to proceed, with continuous agitation for approximately 8 hours.

Post-Reaction Bead Cleanup

Upon completion of the reaction, the reaction suspension is added to a 500 mL separatory funnel and allowed to settle. The aqueous phase is removed and the beads are further washed with 250 mL of deionized water. The beads are then collected by Buchner filtration (Whatman Grade 1 paper) and washed with water and methanol. The beads are placed in a Soxhlet extractor and extracted with acetone. The extracted beads are removed from the thimble and dried at 70° C. The beads are fractionated by size using the appropriate mesh sieve (#16 and #20/50 mesh beads are collected). The beads can be stored dry indefinitely at ambient temperature. CBD-mono beads were used directly for cannabinoid studies or were further activated by hydrolysis of the covalently bound CBD monomer.

### Example 6. Exemplary CBD Absorption Study

1.5 g MIP beads (8% CBD-mono, 5% P123, #16 mesh, KOH hydrolysis) were shaken with a 20 mL solution of CBD (1 mg/mL) in 40/60 water/ethanol at ambient temperature. Aliquots were removed at several time points and assayed using the RP-HPLC method to determine absorption of target compounds into the beads. The RP-HPLC analysis indicated that 8 mg/g was absorbed (12 mg total for 1.5 g) (See FIG. 5).

The beads were then subjected to desorption by 1^{st} washing the beads with 40/60 water/ethanol, followed by incubating with a 20 mL ethanol solution for 18 hours. The solutions were assayed by the RP-HPLC method to determine the amount desorbed form beads.

When an analogous NIP polymer is tested (no surrogate monomer), ˜1-2 mg/g binding of CBD is noted.

### Example 7. Preparation of Surrogate t-THC-03

To a 12000 mL round-bottom flask equipped with overhead mechanical stirrer was added 150.0 g olivetol and 118.4 g trans-4-methylcyclohexanecarboxylic acid and 7500 mL ethyl acetate to dissolve, followed by the sequential addition of 175.4 g of EDC and 2.55 g of DMAP. After the addition was complete, the mixture was stirred until complete no change by silica gel TLC (Rf 0.3 in 10% EtOAc/hexanes, 17 hours). At this time, the mixture was washed with 3000 mL of 1 N aq. HCl, dried over 500 g Na₂SO₄, filtered and concentrated to yield crude product as an oil. Purification by silica gel chromatography (1500 g silica gel, 0-10% EtOAc in hexanes) yielded 67.8 g pure product (27%) as a white solid.

¹H NMR (400 MHz, DMSO-d₆): δ 0.86 (m, 6H), 1.00 (m, 2H), 1.25-1.55 (m, 9H), 1.70 (m, 2H), 2.00 (m, 2H), 2.46 (m, 3H), 6.29 (d, 1H), 6.32 (s, 1H), 6.47 (s, 1H), 9.54 (s, 1H).

¹³C NMR (100 MHz, DMSO-d₆): δ 13.9, 21.9, 22.0, 28.5, 30.8, 31.4, 33.6, 35.0, 39.1, 42.2, 106.2, 112.0, 112.6, 144.6, 151.3, 157.9, 173.8.

### Example 8. Exemplary Synthesis of Macroreticular Beads with t-THC-03 in 50 L Ace Ready Reactor

Preparation of Aqueous Phase

Polyvinyl alcohol (PVOH, average Mw 89,000-98,000, 99+% hydrolyzed, 210 g) is dissolved in water (11150 mL) through gentle heating to 80° C. 93 g of boric acid is dissolved in 2800 mL water and slowly added when the PVOH cools to 50° C. or lower

Preparation of the Organic Phase and Polymerization

129 g of Pluronic P123 (PEG, MW ˜5800) is combined with 1851 mL of styrene, 990 mL of divinylbenzene, 1806 mL of 2-ethylhexanol and 129 g of t-THC-03 in a 10 L pitcher beaker equipped with a stir bar and allowed stir until fully dissolved under ambient conditions. At this time, 26 g of AIBN is added to the solution and dissolved completely. When dissolved, the solution degassed for 10 minutes with nitrogen gas and transferred to a 5 L addition funnel installed on the reactor. The aqueous is added to the 50 L reactor body and degassed for 10 minutes while heating 70° C. with stirring at 250 rpm. When the temperature reaches 70° C., the organic phase is added to the aqueous phase at a rate of 5.5 mL/s. The reaction is allowed to proceed, with continuous stirring at 250 rpm for approximately 8 hours at 70° C. under a nitrogen atmosphere.

Post-Reaction Bead Cleanup

Upon completion of the reaction, the reactor is allowed to cool to ambient temperature and the stirring is stopped. The liquid phase is drained and the residual solid polymer is washed with 2×8 L of water, 1×30 L of water, with the water being drained after each wash. The polymer is removed from the reactor by suspension in adequate water to allow polymer to be flushed out of bottom of reactor after the drain plug is fully removed, leaving a much larger bottom opening. The polymer is vacuum filtered using an 18″ Buchner funnel, washing with 8 L of MeOH, 3×8 L of acetone and aspirated to dryness. The crude beads are suspended in 5 L of acetone, agitated for 2 min and then filtered through a 200 micron mesh filter. The process is repeated 1×5 L of acetone and 2×4 L of ethanol. After solvent has drained from last wash, the beads are dried in an 18″ Buchner funnel under vacuum. The beads are fractionated by size using the appropriate mesh sieves (#20 (1260 g), #35 (728 g) and #50 (446 g) mesh beads are collected). The beads can be stored dry indefinitely at ambient temperature.

### Example 9. Selectivity for THC/CBD Using Unactivated CBD Mono Surrogate Polymer Beads

Bead Preparation: 0.30 g of dried polymer beads (CBD-mono, unactivated) were suspended in 60% ethanol/water overnight to fully wet. The solvent was decanted before use.

CBD/THC Stock solution: A 5 mL stock solution of CBD isolate (Extract Labs, 99%) and THC (Sigma-Aldrich Cat #T4764) was prepared in a 1:1 ratio (w/w) at a total cannabinoid concentration of 2 mg/mL in 60% ethanol/water.

Absorption of CBD/THC on beads: The CBD-mono beads previously wetted in 60% ethanol/water were incubated with 4 mL of the CBD/THC stock solution at ambient temperature with shaking for a total of 21 h.

RP-HPLC analysis of CBD/THC pregnant solution: Aliquots were removed at several time points (1, 2, 4, 6 and 21 hours) and assayed using the RP-HPLC method to determine absorption of target compounds into the beads (see FIG. 6). The RP-HPLC analysis indicated that both CBD and THC were absorbed by the beads, with a preference of ˜1.4:1 for THC over CBD.

### Example 10. Capacity of CBD-Mono Beads and t-THC-03 Beads

Instrument: A BioRad Econo Chromatography System with a BioRad Econo peristaltic pump, BioRad Econo UV Monitor, Isco Foxy Jr fraction collector, and a BioRad Econo column (1 cm×20 cm). Loading, washing and elution of cannabinoids was performed at a flow rate of 1 mL/min. Samples were collected for every 0.5 CV. Analysis of sample fractions was performed using a Shimadzu UV-1201 UV/Vis spectrophotometer to measure absorption at 280 nm wavelength.

Capacity of CBD-Mono Beads

Column Preparation: 3 g of dried polymer (CBD-mono, unactivated) was suspended in Ethanol overnight to fully wet. The polymer beads were then slurry packed into a BioRad Econo column (1 cm×20 cm) to provide a column height of 15 cm (12 mL, 1 CV). The column was eluted with ethanol at a flow rate of 1 mL/min until UV absorbance stabilized. The column was then eluted with 60% ethanol/water (1 mL/min) until equilibrated as determined by UV absorbance (˜3 CV).

CBD Stock solution: A solution of CBD isolate (Extract Labs, 99%) was prepared at a concentration of 1 mg/mL in 60% ethanol/water.

Absorption of CBD on Column: The CBD stock solution was loaded on to the column at a flow rate of 1 mL/min. A total of 2.33 CV (28 mL, 28 mg of CBD) was loaded onto column. Fractions of eluted material were collected at 6 mL total volume (0.5 CV).

Column wash: The column was washed with 60% ethanol for 8 CV with fractions collected at 0.5 CV)

CBD elution: The column was eluted with ethanol to desorb CBD from the beads with fractions collected at 0.5 CV intervals. A total of 3 CV of eluted materials were collected.

Capacity determination: Collected fractions (load, wash, elution) were analyzed for CBD by UV/Vis absorption at 280 nm. The total amount of CBD retained was determined to be 24 mg (8 mg/g) based on the UV/Vis analysis of the eluted fractions. The load and wash fractions did not display any absorbance (see FIG. 7).

Capacity of t-THC-03 Beads

Column Preparation: 3 g of dried polymer beads (t-THC-03, #50 mesh) were suspended in ethanol overnight to fully wet. The polymer beads were then slurry packed into a BioRad Econo column (1 cm×20 cm) to provide a column height of 15.5 cm (12 mL, 1 CV). The column was eluted with ethanol at a flow rate of 1 mL/min until UV absorbance stabilized. The column was then eluted with 58% ethanol/water (1 mL/min) until equilibrated as determined by UV absorbance (˜3 CV).

CBD Stock solution: A solution of CBD isolate (Aerosource H, LLC, 99%) was prepared at a concentration of 1 mg/mL in 58% ethanol/water.

Absorption of CBD on Column: The CBD stock solution was loaded on to the column at a flow rate of 1 mL/min. A total of 4.5 CV (54 mL, 54 mg of CBD) was loaded onto column. Fractions of eluted material were collected at 6 mL total volume (0.5 CV).

Column wash: The column was washed with 58% ethanol for 3CV with fractions collected at 0.5 CV)

CBD elution: The column was eluted with ethanol to desorb CBD from the beads with fractions collected at 0.5 CV intervals. A total of 7 CV of eluted materials were collected.

Capacity determination: Collected fractions (load, wash, elution) were analyzed for CBD by UV/Vis absorption at 280 nm. The total amount of CBD retained was determined to be 42 mg (14 mg/g) based on the UV/Vis analysis of the eluted fractions. The load and wash fractions did not display any absorbance.

### Example 11. Absorption and Elution of THC/CBD from Unactivated CBD-Mono Polymer Beads

Column Preparation: 1.5 g of dried polymer (CBD-mono, unactivated) were suspended in ethanol overnight to fully wet. The polymer beads were then slurry packed into a BioRad Econo column (1 cm×20 cm) to provide a column height of 8 cm (6.3 mL, 1 CV). The column was eluted with ethanol at a flow rate of 1 mL/min until UV absorbance stabilized. The column was then eluted with 60% ethanol/water (1 mL/min) until equilibrated as determined by UV absorbance (˜3 CV).

CBD/THC Stock solution: A solution of CBD isolate (Extract Labs, 99%) and THC (Sigma-Aldrich Cat #T4764) was prepared in a 4:1 ratio (w/w) at a total cannabinoid concentration of 1 mg/mL in 60% ethanol/water.

Absorption of CBD/THC on column: The CBD/THC stock solution was loaded on to the column at a flow rate of 1 mL/min. A total of 2 CV (13 mL, 13 mg of cannabinoids) was loaded onto column (no breakthrough was detected). Fractions of eluted material were collected at 3.25 mL total volume (0.5 CV).

Column wash: The column was washed with 60% ethanol for 3 CV with fractions collected at 0.5 CV).

CBD elution: The column was eluted with ethanol to desorb CBD/THC from the beads with fractions collected at 0.5 CV intervals. A total of 3 CV of eluted materials were collected.

Recovery determination: Collected fractions (load, wash, elution) were analyzed for CBD and THC by RP-HPLC analysis. The analysis of the stock solution (FIG. 8A) and the pooled ethanol elution fractions (FIG. 8B) were identical indicating that both cannabinoids are absorbed/desorbed in a similar manner. Individual elution fraction analysis suggests that CBD elutes in slight preference to THC under the current conditions.

### Example 12. Selective Recovery of Cannabinoids from Hemp Distillate Using CBD-Mono Beads

Column Preparation: 125 g of dried polymer (CBD-mono, unactivated, #50 mesh) was suspended in ethanol overnight to fully wet. The polymer beads were then slurry packed equally into 2 BioRad Econo columns (2.5 cm×50 cm) connected in series to provide a total column height of 90 cm (450, 1 CV). The column was then eluted with 62% ethanol/water (18 mL/min) until equilibrated.

Hemp Distillate Stock solution: A 100 mL solution of hemp distillate (65% CBD, 3% THC and 5% CBC were major components) was prepared at a total concentration of 20 mg/mL in 62% ethanol/water.

Absorption of Hemp Distillate on Column: The distillate stock solution was loaded on to the column at a flow rate of 18 mL/min. A total of 0.22 CV (100 mL, 2 g of distillate) was loaded onto column. Fractions of loaded material were collected at ˜225 mL total volume (0.5 CV).

Column elution: The column was eluted with 5CV each of 58% ethanol/water, 62% ethanol/water, 67% ethanol/water, respectively with fractions collected at 0.5 CV (˜225 mL) intervals. A total of 15 CV of eluted materials were collected.

Recovery determination: Eluted fractions were analyzed for CBD and THC by RP-HPLC analysis. The analysis indicated that THC was only present in the 67% ethanol/water elution fractions 5CV each of 58% ethanol water and 62% ethanol/water fractions were pooled along with the 1^{st} two CV of 67% ethanol/water fractions. The RP-HPLC analysis of these materials indicated a total of 585 mg of CBD containing 0.3% THC were recovered. This corresponds to a yield of 50% recovered CBD based on the total amount of CBD only in the original distillate. 5CV each of 58% ethanol water and 62% ethanol/water fractions, respectively, were pooled and analyzed for CBD/THC content (Fraction 1). Subsequently, the 1^{st} two CV of 67% ethanol/water fractions were combined with the THC free fractions and re-analyzed for CBD-THC content (Fraction 2). The results of the RP-HPLC analysis of these materials indicated is summarized below.

| | CBD | THC | % THC | |
|---|---|---|---|---|
| Material | (mg) | (mg) | w/w | % CBD Recovery |
| Hemp Distillate | 1300 | 1.5 | 3% (of | NA |
| (2 g total mass) | | | total mass) | |
| Fraction 1 | 389 | ND | | 30 % (based on |
| | | | | total CBD) |
| Fraction 2 | 585 | 1.7 | 0.3 | 50% (based on |
| | | | | total CBD) |

### Example 13: Selective Recovery of Cannabinoids from Hemp Distillate Using t-THC-03 Beads

Column Preparation: 3 g of dried polymer (t-THC-03 beads, #50 mesh) was suspended in ethanol overnight to fully wet. The polymer beads were then slurry packed into a BioRad Econo column (1 cm×20 cm) to provide a column height of 15.5 cm (12 mL, 1 CV). The column was eluted with ethanol at a flow rate of 1 mL/min until UV absorbance stabilized. The column was then eluted with 58% ethanol/water (1 mL/min) until equilibrated as determined by UV absorbance (˜3 CV).

Hemp Distillate Stock-solution: A 100 mL solution of hemp distillate (65% CBD, 3% THC and 5% CBC were major components) was prepared at a total concentration of 1.4 mg/mL in 58% ethanol/water and filtered with a 0.7μ membrane filter.

Absorption of Hemp Distillate on Column: The distillate stock solution was loaded on to the column at a flow rate of 1 mL/min. A total of 3 CV (36 mL, 50.4 mg of distillate) was loaded onto column. Fractions of loaded material were collected at 6 mL total volume (0.5 CV).

Column elution: The column was eluted with 3CV each of 58% ethanol/water, 65% ethanol/water, 75% ethanol/water and 100% ethanol, respectively with fractions collected at 0.5 CV (6 mL) intervals. A total of 15 CV of eluted materials were collected.

Recovery determination: Eluted fractions were analyzed for CBD and THC by RP-HPLC analysis. The analysis indicated that CBD began to elute in the 65% ethanol/water fractions and THC was not detected until the 3^{rd} CV of the 75% ethanol/water elution fractions. With ethanol all cannabinoids were eluted. The 3CV of 65% ethanol/water and 1^{st} 2.5CV of the 75% ethanol/water fractions were pooled and analyzed for CBD/THC content (Fraction 1). Subsequently, the final 0.5 CV of 75% ethanol/water and the initial 1.5 CV of the 100% ethanol elution were combined with the THC free fractions and re-analyzed for CBD-THC content (Fraction 2). The results of the RP-HPLC analysis of these materials indicated is summarized below.

| | CBD | THC | | % THC | |
|---|---|---|---|---|---|
| Material | (mg) | (mg) | | w/w | % CBD Recovery |
| Hemp | 32.8 mg | 1.5 | mg | 3% (of | NA |
| Distillate | | | | total mass) | |
| (50.4 mg | | | | | |
| total mass) | | | | | |
| Fraction 1 | 3.1 mg | ND | | | 9.5% (based on |
| | | | | | total CBD) |
| Fraction 2 | 12.5 mg | 0.039 | mg | 0.31% | 38% (based on |

total CBD)

### EMBODIMENTS

1. A plurality of macroreticular polymer beads comprising a copolymer having a plurality of complexing cavities that selectively bind a target cannabinoid, wherein the copolymer comprises:
   (a) a cannabinoid surrogate-containing monomer,
   (b) a non-crosslinking monomer, and
   (c) a crosslinking monomer;
   wherein the cannabinoid surrogate has substantially the same steric and functional properties of the target cannabinoid and
   wherein the cannabinoid surrogate is removed to provide a complexing cavity that selectively binds a target cannabinoid.
2. A plurality of macroreticular polymer beads comprising a copolymer that selectively binds a target cannabinoid, wherein the copolymer comprises:
   (a) a cannabinoid surrogate-containing monomer,
   (b) a non-crosslinking monomer, and
   (c) a crosslinking monomer;
   wherein the cannabinoid surrogate has substantially the same steric and functional properties of the target cannabinoid.
3. The macroreticular polymer beads of any one of embodiments 1-2, wherein the cannabinoid surrogate-containing monomer has the formula:

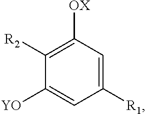

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, benzoyl, 4-vinylbenzoyl, 2-vinylbenzoyl, 3-vinylbenzoyl, allyl, methacryloyl, acryloyl, carbamoyl, glycidyl methacrylate, and glycidyl acrylate;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group;
wherein the cannabinoid surrogate has substantially the same steric and functional properties of a target cannabinoid and
wherein at least one of X and Y contains a polymerizable group.
4. The macroreticular polymer beads of embodiment 3, wherein R₂ is the cannabinoidyl group and has the formula:

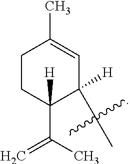

5. The macroreticular polymer beads of embodiment 3, wherein the cannabinoid surrogate-containing monomer has the formula:

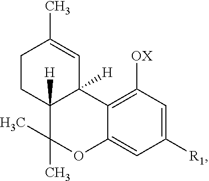

wherein
X contains a polymerizable group.
6. The macroreticular polymer beads of any one of embodiments 1-5, wherein the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.
7. The macroreticular polymer beads of any one of embodiments 1-2, wherein the cannabinoid surrogate-containing monomer is selected from the group consisting of:

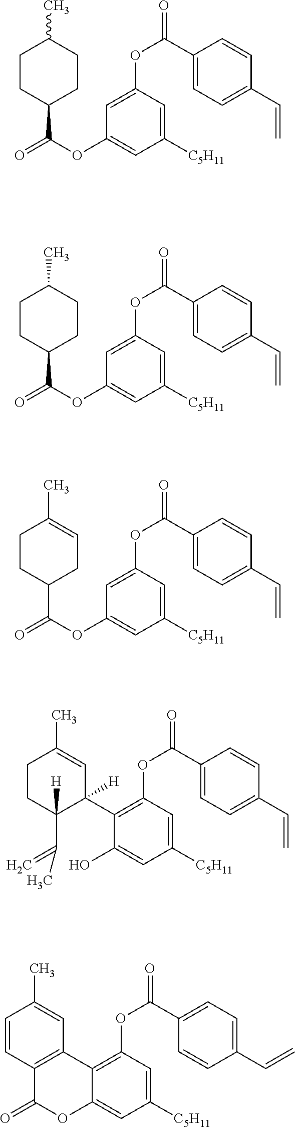

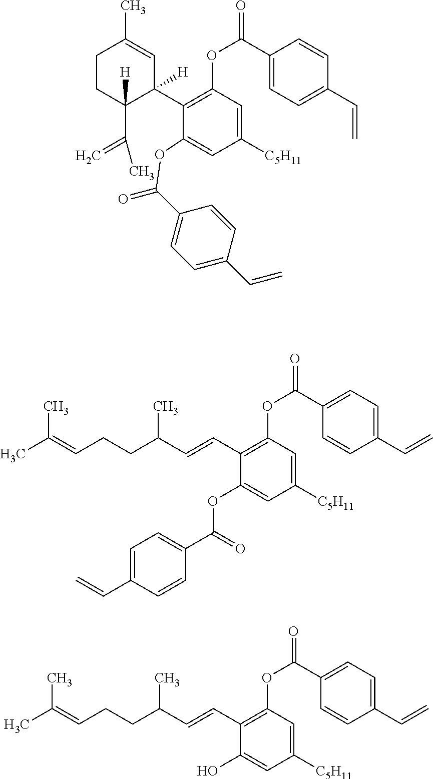

8. A plurality of macroreticular polymer beads comprising a copolymer having a plurality of complexing cavities which selectively bind a target cannabinoid, wherein the copolymer is prepared from:
   (a) a cannabinoid surrogate,
   (b) a non-crosslinking monomer, and
   (c) a crosslinking monomer:
   wherein the cannabinoid surrogate has substantially the same steric and functional properties of the target cannabinoid and
   wherein the cannabinoid surrogate is removed to provide a complexing cavity that selectively binds a target cannabinoid.
9. The macroreticular polymer beads of embodiment 8, wherein the cannabinoid surrogate has the formula:

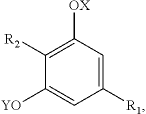

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, and benzoyl;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid.
10. The macroreticular polymer beads of embodiment 9, wherein the cannabinoid surrogate is selected from the group consisting of:

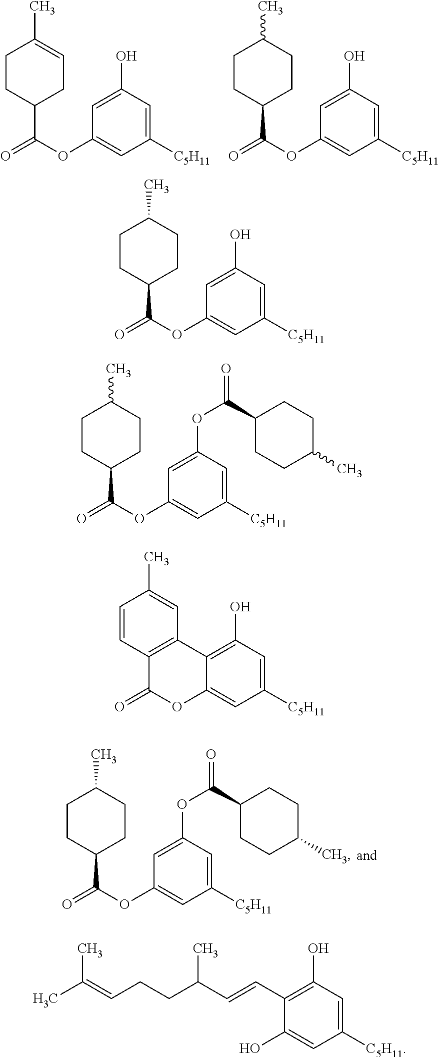

11. The macroreticular polymer beads of any one of embodiments 1-10, wherein the target cannabinoid is selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄(CBD-C₄), cannabidivarinic acid (CBDVA), cannabidivarin (CBDV), cannabidiorcol (CBD-C₁), tetrahydrocannabinolic acid A (THCA-A), tetrahydrocannabinolic acid B (THCA-B), tetrahydrocannabinolic acid (THCA), tetrahydrocannabinol (THC), tetrahydrocannabinolic acid C₄(THCA-C₄), tetrahydrocannabinol C₄(THC-C₄), tetrahydrocannabivarinic acid (THCVA), tetrahydrocannabivarin (THCV), tetrahydrocannabiorcolic acid (THCA-C₁), tetrahydrocannabiorcol (THC-C₁), Δ⁷-cis-iso-tetrahydrocannabivarin, Δ⁸-tetrahydrocannabinolic acid (Δ8-THCA), cannabivarinodiolic (CBNDVA), cannabivarinodiol (CBNDV), Δ⁸-tetrahydrocannabinol (Δ⁸-THC), Δ⁹-tetrahydrocannabinol (Δ⁹-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabivarinselsoin (CBEV), cannabivarinselsoinic acid (CBEVA), cannabielsoic acid (CBEA), cannabielvarinsoin (CBLV), cannabielvarinsoinic acid (CBLVA), cannabinolic acid (CBNA), cannabinol (CBN), cannabivarinic acid (CBNVA), cannabinol methylether (CBNM), cannabinol-C₄ (CBN-C₄), cannabivarin (CBV), cannabino-C₂ (CBN-C₂), cannabiorcol (CBN-C₁), cannabinodiol (CBND), cannabinodiolic acid (CBNDA), cannabinodivarin (CBDV), cannabitriol (CBT), 10-ethoxy-9-hydroxy-Δ^{6a}-tetrahydrocannabinol, 8,9-dihydroxy-Δ^{6a(10a)}-tetrahydrocannabinol (8,9-Di-OH-CBT-C₅), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ^{6a(10a)}-tetrahydrocannabinol (MEC), Δ⁹-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, and dodeca-2E,4E-dienoic acid isobutylamide.
12. The macroreticular polymer beads of embodiment 11, wherein the target cannabinoid is CBD.
13. The macroreticular polymer beads of embodiment 11, wherein the target cannabinoid is CBDA.
14. The macroreticular polymer beads of embodiment 11, wherein the target cannabinoid is THC.
15. The macroreticular polymer beads of embodiment 11, wherein the target cannabinoid is THCA.
16. The macroreticular polymer beads of embodiment 11, wherein the target cannabinoid is selected from the group consisting of CBG, CBN, CBC, and THCV.
17. The macroreticular polymer beads of any one of embodiments 1-16, wherein the crosslinking monomer is selected from the group consisting of alkylene glycols and polyalkylene glycol diacrylates, polyalkylene glycol methacrylates, vinyl acrylates, vinyl methacrylates, allyl acrylates or allyl methacrylates, divinylbenzene, diallyldiglycol dicarbonate, diallyl maleate, diallyl fumarate, diallyl itaconate, vinyl esters, the dimethacrylates or diacrylates of bis-phenol A or ethoxylated bis-phenol A, methylene bisacrylamide, polymethylene bisacrylamide, bismuth-acrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1, 1-dimethy 1-2-isocyanatoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12, 17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; and divinyl tin dichloride.
18. The macroreticular polymer beads of embodiment 17 wherein the crosslinking monomer is divinylbenzene.
19. The macroreticular polymer beads of any one of embodiments 1-18, wherein the non-crosslinking monomer is selected from the group consisting of methylmethacrylate, other alkyl methacrylates, alkylacrylates, allyl or aryl acrylates and methacrylates, cyanoacrylate, styrene, substituted styrenes, methyl styrene (multisubstituted) including 1-methylstyrene; 3-methylstyrene; 4-methylstyrene, etc.; vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy) ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; aryloyl chloride; l-α-acryloyloxy-β,β-dimethyl-γ-butyrolactone; N-acryloxy succinimide acryloxytris(hydroxymethyl)amino-methane; N-acryloyl chloride; N-acryloyl pyrrolidinone; N-acryloyl-tris(hydroxymethyl)amino methane; 2-aminoethyl methacrylate; N-(3-aminopropyl)methacrylamide; (o, m, or p)-amino-styrene; t-amyl methacrylate; 2-(1-aziridinyl)ethyl methacrylate; 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; -bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; t-butylacrylamide; butyl acrylate; butyl methacrylate; (o, m, p)-bromo styrene; t-butyl acrylate; 1-carvone; (S)-carvone; (−)-carvyl acetate; 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 1-(3-butenyl)-4-vinylbenzene; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethy 1-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); glycidyl acrylate, glycidyl methacrylate; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; (±)-linalool; linalyl acetate; p-mentha-1,8-diene; p-mentha-6,8-dien-2-ol; methyleneamino acetonitrile; methacrolein; [3-(methacryloylamino)-propyl] trimethylammonium chloride; methacrylamide; methacrylic acid; methacrylic anhydride; methacrylonitrile; methacryloyl chloride; 2-(methacryloyloxy)ethyl acetoacetate; (3-meth-acryloxypropyl)trimethoxy silane; 2-(methacryloxy)ethyl trimethylammonium methylsulfate; 2-methoxy propene (isopropenyl methyl ether); methyl-2-(bromomethyl)acrylate; 5-methyl-5-hexen-2-one; methyl methacrylate; N,N′methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; methyl vinyl ether; methyl-2-vinyloxirane; methyl vinyl sulfone; 4-methyl-5-vinylthiazole; myrcene; t-nitrostyrene; 3-nitrostyrene; 1-nonadecene; 1,8-nonadiene; 1-octadecene; 1, 7-octadiene; 7-82ctane-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyanoethylene; trans 3-chloroacrylic acid; 2-trifluoromethyl propene; 2-(trifluoromethyl)propenoic acid; 2,4,4′-trimethyl-1-pentene; 3, 5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethylsulfone; 4-(vinyl benzyl)-2-chloroethyl sulfone; N-(p-vinylbenzyl)-N,N′-dimethyl amine; 4-vinyl biphenyl (4-phenylstyrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl peroxcyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinylquinoline; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinylquinoline; 1-vinyl silatrane; vinyl sulfone; vinyl sulfonic acid sodium salt; a-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy) silane; vinyl 2-valerate; vinyl benzoic acid; vinyl imidazole; vinylpyridine; vinylbenzylamine; hydroxyethylmethacrylate (HEMA); and aminopropylmethacrylate.
20. The macroreticular polymer beads of embodiment 19, wherein the non-crosslinking monomer is styrene.
21. The macroreticular polymer beads of any one of embodiments 1-20, wherein the non-crosslinking monomer is styrene and the crosslinking monomer is divinylbenzene.
22. The macroreticular polymer beads of any one of embodiments 1-21, wherein the beads have a selectivity coefficient for the target cannabinoid of greater than about 10.
23. The macroreticular polymer beads of any one of embodiments 1-22, having a surface area of about 0.1-500 m²/g.
24. The macroreticular polymer beads of any one of embodiments 1-23, having an average particle size ranging from about 250 μm to about 1.5 mm.
25. A method of preparing macroreticular molecularly imprinted polymer beads comprising: (1) polymerizing:
   (a) a cannabinoid surrogate-containing monomer,
   (b) a non-crosslinking monomer, and
   (c) a crosslinking monomer;
   wherein the cannabinoid surrogate has substantially the same steric and functional properties of the target cannabinoid and
   (2) removing the cannabinoid surrogate to provide a complexing cavity that selectively binds a target cannabinoid.
26. A method of preparing a macroreticular molecularly imprinted polymer that selectively binds a target cannabinoid, the method comprising:
   (1) polymerizing:
      (a) a cannabinoid surrogate-containing monomer,
      (b) a non-crosslinking monomer, and
      (c) a crosslinking monomer;
   wherein the cannabinoid surrogate has substantially the same steric and functional properties of the target cannabinoid.
27. The method of any one of embodiments 25-26, wherein the cannabinoid surrogate-containing monomer has the formula:

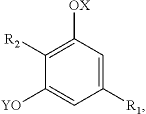

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, benzoyl, 4-vinylbenzoyl, 2-vinylbenzoyl, 3-vinylbenzoyl, allyl, methacryloyl, acryloyl, carbamoyl, glycidyl methacrylate, and glycidyl acrylate;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid and
wherein at least one of X and Y contains a polymerizable group.
28. The method of embodiment 27, wherein R₂ is the cannabinoidyl group has the formula:

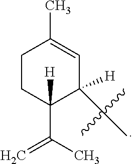

29. The method of embodiment 27, wherein the cannabinoid surrogate-containing monomer has the formula:

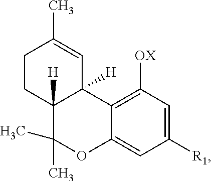

wherein
X contains a polymerizable group.
30. The method of any one of embodiments 25-29, wherein the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.
31. The method of any one of embodiments 25-26, wherein the cannabinoid surrogate-containing monomer is selected from the group consisting of:

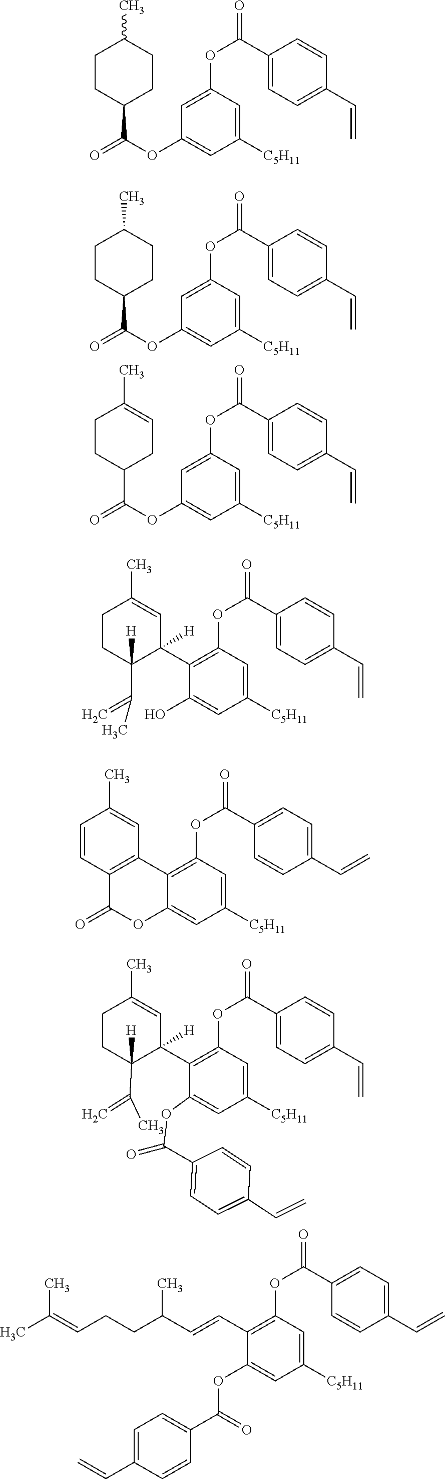

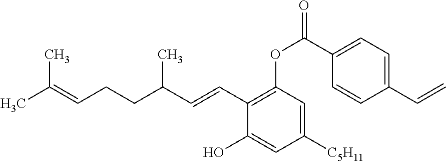

32. A method of preparing macroreticular molecularly imprinted polymer heads, the method comprising:
   (1) polymerizing:
      (a) cannabinoid surrogate,
      (b) a non-crosslinking monomer, and
      (c) a crosslinking monomer:
      wherein the cannabinoid surrogate has substantially the same steric and functional properties of the target cannabinoid and
   (2) removing the cannabinoid surrogate to provide a complexing cavity that selectively binds a target cannabinoid.
33. The method of embodiment 32, wherein the cannabinoid surrogate has the formula:

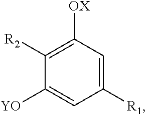

wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, and benzoyl;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid.
34. The method of embodiment 32, wherein the cannabinoid surrogate is selected from the group consisting of:

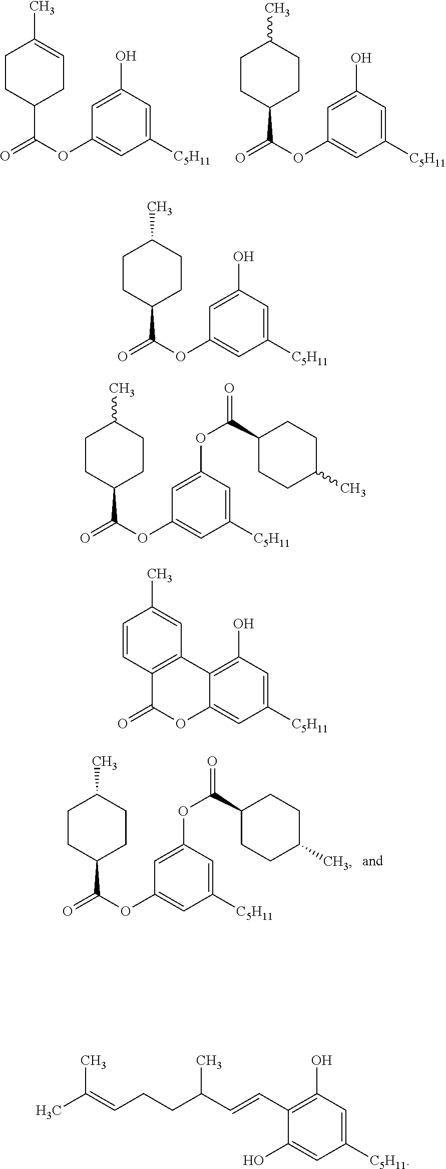

35. The method of any one of embodiments 25-34, wherein the non-crosslinking monomer is styrene.
36. The method of any one of embodiments 25-35, wherein the crosslinking monomer is divinylbenzene.
37. The method of any one of embodiments 25-36, wherein the non-crosslinking monomer is styrene and the crosslinking monomer is divinylbenzene.
38. The method of any one of embodiments 25-37, wherein polymerizing occurs in the presence of a pore forming additive.
39. The macroreticular molecularly imprinted polymer beads prepared according to any one of embodiments 25 and 27-38.
40. The macroreticular molecularly imprinted polymer prepared according to any one of embodiments 26-38.
41. A method of selectively sequestering one or more target cannabinoids from a solution of one or more target cannabinoids admixed with other cannabinoids, the method comprising:
   first contacting the macroreticular polymer beads of any one of embodiments 1 and 3-24 with a hydrolyzing solution, whereby the cannabinoid surrogates are removed from the macroreticular polymer beads, then contacting the stripped beads with the cannabinoid-containing solution, thereby selectively sequestering the target cannabinoid in the macroreticular polymer beads.
42. A method of selectively sequestering one or more target cannabinoids from a solution of one or more target cannabinoids admixed with other cannabinoids, the method comprising:
   contacting the macroreticular molecularly imprinted polymer of any one of embodiments 2-24 with the cannabinoid-containing solution, thereby selectively sequestering the target cannabinoid in the macroreticular polymer beads.
43. The method of any one of embodiments 41-42, further comprising stripping the target cannabinoid from the macroreticular polymer beads.
44. A method of removing THC from a cannabinoid-containing solution comprising THC and CBD, the method comprising:
   (1) contacting the solution with the macroreticular polymer beads of any one of embodiments 1-24, whereby the THC is selectively complexed in the beads, and
   (2) recovering a solution comprising CBD that is substantially THC-free.
45. The method of embodiment 44, further comprising stripping the beads with a stripping solution, whereby the THC is substantially removed from the beads.
46. A method of removing at least two target cannabinoids from a solution containing at least two target cannabinoids admixed with other cannabinoids, the method comprising:
   (1) providing an activated form of the macroreticular polymer beads of any one of embodiments 1, and 3-24 having an affinity for a first target cannabinoid;
   (2) contacting the solution with the macroreticular polymer beads, whereby the first target cannabinoid is selectively complexed in the beads,
   (3) recovering a solution that is substantially free of the first target cannabinoid; and
   (4) providing an activated form of the macroreticular polymer beads of any one of embodiments 1, and 3-24, having an affinity for a second target cannabinoid in the recovered solution;
   (5) contacting the solution of Step (3) with the macroreticular polymer beads, whereby the second target cannabinoid is selectively complexed in the beads, and
   (6) recovering a solution that is substantially free of the first and second target cannabinoids.
47. A method of removing at least two target cannabinoids from a solution containing at least two target cannabinoids admixed with other cannabinoids, the method comprising:
   (1) providing the macroreticular polymer beads of any one of embodiments 2-24 having an affinity for a first target cannabinoid;
   (2) contacting the solution with the macroreticular polymer beads, whereby the first target cannabinoid is selectively complexed in the macroreticular molecularly imprinted polymer beads,
   (3) recovering a solution that is substantially free of the first target cannabinoid; and
   (4) providing the macroreticular molecularly imprinted polymer beads of any one of embodiments 2-24, having an affinity for a second target cannabinoid in the recovered solution;
   (5) contacting the solution of Step (3) with the macroreticular polymer beads, whereby the second target cannabinoid is selectively complexed in the beads, and
   (6) recovering a solution that is substantially free of the first and second target cannabinoids.
48. The method of embodiment 46 or 47, further comprising repeating the method until substantially all target cannabinoids are removed.
49. The method of embodiment 48, further comprises repeating the method 1-10 more times.
50. The method of any one of embodiments 46-49, wherein the target cannabinoid is selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), tetrahydrocannabinolic acid A (THCA-A), tetrahydrocannabinolic acid B (THCA-B), tetrahydrocannabinolic acid (THCA), tetrahydrocannabinol (THC), tetrahydrocannabinolic acid C₄(THCA-C₄), tetrahydrocannabinol C₄ (THC-C₄), tetrahydrocannabivarinic acid (THCVA), tetrahydrocannabivarin (THCV), tetrahydrocannabiorcolic acid (THCA-C₁), tetrahydrocannabiorcol (THC-C₁), Δ⁷-cis-iso-tetrahydrocannabivarin, Δ⁸-tetrahydrocannabinolic acid (Δ8-THCA), cannabivarinodiolic (CBNDVA), cannabivarinodiol (CBNDV), Δ⁸-tetrahydrocannabinol (Δ⁸-THC), Δ⁹-tetrahydrocannabinol (Δ⁹-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabivarinselsoin (CBEV), cannabivarinselsoinic acid (CBEVA), cannabielsoic acid (CBEA), cannabielvarinsoin (CBLV), cannabielvarinsoinic acid (CBLVA), cannabinolic acid (CBNA), cannabinol (CBN), cannabivarinic acid (CBNVA), cannabinol methylether (CBNM), cannabinol-C₄ (CBN-C₄), cannabivarin (CBV), cannabino-C₂ (CBN-C₂), cannabiorcol (CBN-C₁), cannabinodiol (CBND), cannabinodiolic acid (CBNDA), cannabinodivarin (CBDV), cannabitriol (CBT), 10-ethoxy-9-hydroxy-Δ^{6a}-tetrahydrocannabinol, 8,9-dihvdroxy-Δ^{6a(10a)}-tetrahydrocannabinol (8,9-Di-OH-CBT-C₅), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ^{6a(10a)}-tetrahydrocannabinol (MEC), Δ⁹-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, and dodeca-2E,4E-dienoic acid isobutylamide.
51. The method of embodiment 50, wherein the target cannabinoids are THC and CBD.
52. A method of removing THC from a cannabinoid-containing solution comprising THC and CBD, the method comprising:
   (1) contacting the solution with the macroreticular polymer beads of any one of embodiments 1-24, whereby the THC and CBD are complexed in the beads;
   (2) selectively stripping CBD from the macroreticular polymer beads; and
   (3) recovering a stripped solution comprising CBD that is substantially THC-free
53. A method of purifying a cannabinoid-containing solution, the method comprising:
   (1) contacting the solution with the macroreticular polymer beads of any one of embodiments 1-24, whereby the cannabinoids in the solution are complexed in the beads and
   (2) stripping the cannabinoids from the macroreticular polymer beads to provide a purified cannabinoid-containing solution.
54. The method of embodiment 53, wherein the purified cannabinoid-containing solution contains less than about 0.1 ppm of pesticides.
55. The method of any one of embodiments 53-54, wherein the purified cannabinoid-containing solution contains less than about 10 ppm of heavy metals.
56. The method of any one of embodiments 41-55, wherein the solution is a Cannabis or hemp extract.
57. The method of embodiment 56, wherein the Cannabis or hemp plant extract components are semi-processed.
58. The method of any one of embodiments 41-57 wherein the solution is prepared by:
   i) providing cannabinoid containing plant trim;
   ii) extracting the plant trim with an extraction solvent to provide an extract; and
   iii) optionally winterizing and/or filtering and/or distilling the extract to provide the solution.
59. The method of embodiment 58, wherein the extraction solvent is an alcohol/water mixture.
60. The method of any one of embodiments 46-59, further comprising stripping the target cannabinoid complexed beads or polymer with a stripping solution, whereby the target cannabinoid is substantially removed from the beads.
61. The method of embodiment 43, 45, or 60, wherein the stripping solution is an alcohol or an alcohol/water mixture.
62. The method of embodiment 61, wherein the stripping solution is 50-70% alcohol/water.
63. The method of any one of embodiments 59-62, wherein the alcohol is selected from the group consisting of methanol, ethanol and isopropanol.
64. The method of embodiment 43, 45, or 60, wherein stripping provides substantially pure target cannabinoid.
65. The method of embodiment 64, wherein the target cannabinoid is CBD and stripping provides a substantially pure CBD comprising <1% THC w/w.
66. The method of embodiment 65, wherein stripping provides a substantially pure CBD comprising ≤0.3% THC w/w.
67. The method of embodiment 65 or 66, wherein stripping provides a substantially pure CBD comprising <0.09% THC w/w.
68. A compound selected from the group consisting of:

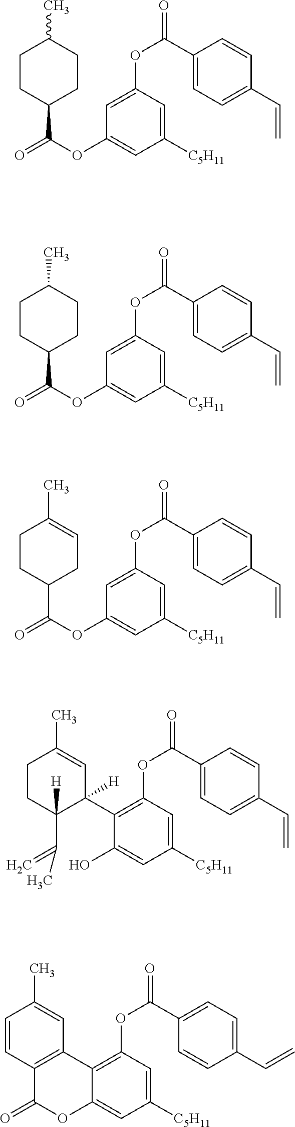

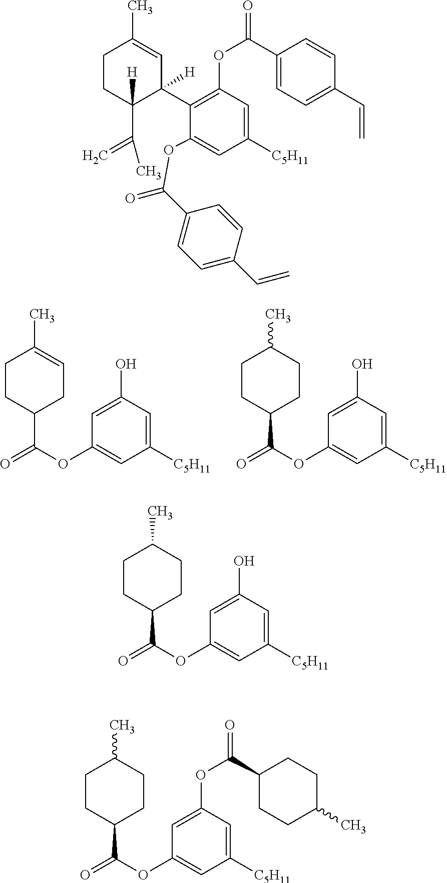

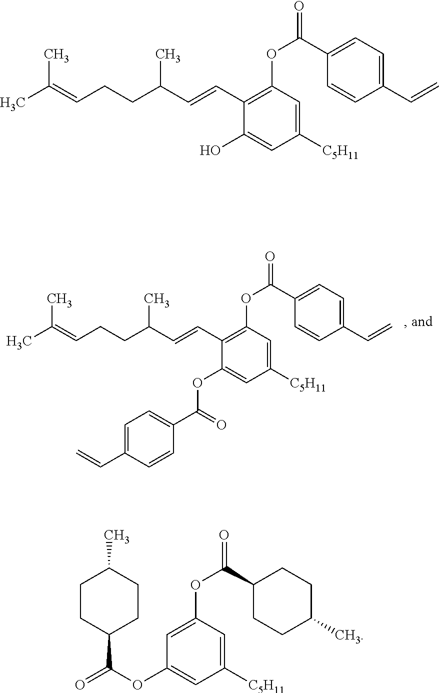

What is claimed is:

1. A plurality of macroreticular polymer beads comprising a copolymer having a plurality of complexing cavities that selectively bind a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid and
wherein the cannabinoid surrogate is removed to provide a complexing cavity that selectively binds a target cannabinoid.

2. A plurality of macroreticular polymer beads comprising a copolymer that selectively binds a target cannabinoid, wherein the copolymer comprises:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid.

3. The macroreticular polymer beads of any one of claims 1-2, wherein the cannabinoid surrogate-containing monomer has the formula: 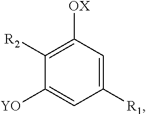
wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, benzoyl, 4-vinylbenzoyl, 2-vinylbenzoyl, 3-vinylbenzoyl, allyl, methacryloyl, acryloyl, carbamoyl, glycidyl methacrylate, and glycidyl acrylate;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group;
wherein the cannabinoid surrogate has substantially the same steric and functional properties of a target cannabinoid and
wherein at least one of X and Y contains a polymerizable group.

4. The macroreticular polymer beads of claim 3, wherein R₂ is the cannabinoidyl group and has the formula:

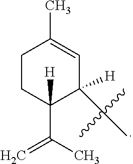

5. The macroreticular polymer beads of claim 3, wherein the cannabinoid surrogate-containing monomer has the formula: 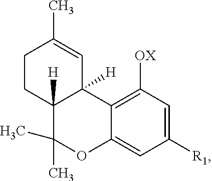
wherein
X contains a polymerizable group.

6. The macroreticular polymer beads of any one of claims 1-5, wherein the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.

7. The macroreticular polymer beads of any one of claims 1-2, wherein the cannabinoid surrogate-containing monomer is selected from the group consisting of:

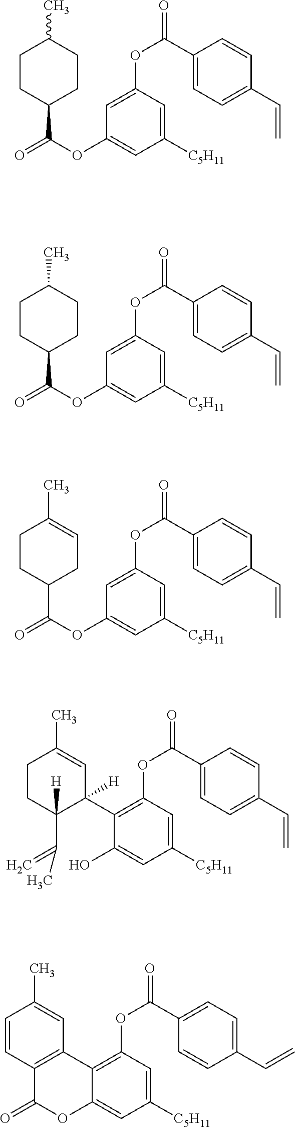

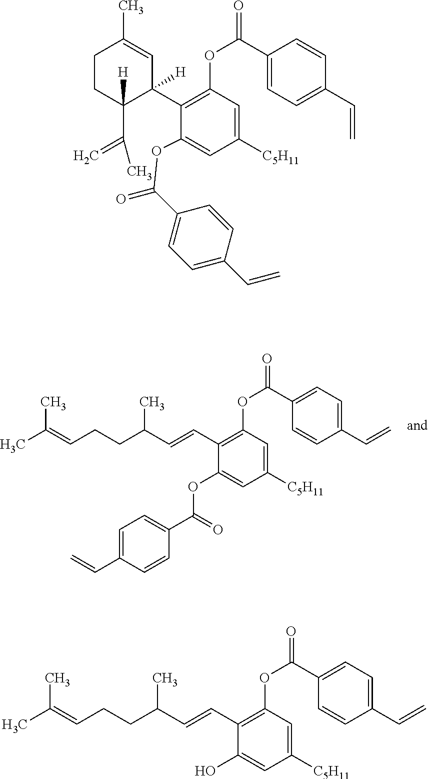

8. A plurality of macroreticular polymer beads comprising a copolymer having a plurality of complexing cavities which selectively bind a target cannabinoid, wherein the copolymer is prepared from:
(a) a cannabinoid surrogate,
(b) a non-crosslinking monomer, and
(c) a crosslinking monomer:
wherein the cannabinoid surrogate has substantially the same steric and functional properties of the target cannabinoid and
wherein the cannabinoid surrogate is removed to provide a complexing cavity that selectively binds a target cannabinoid.

9. The macroreticular polymer beads of claim 8, wherein the cannabinoid surrogate has the formula: 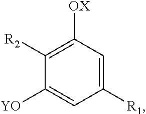
wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, and benzoyl;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid.

10. The macroreticular polymer beads of claim 9, wherein the cannabinoid surrogate is selected from the group consisting of:

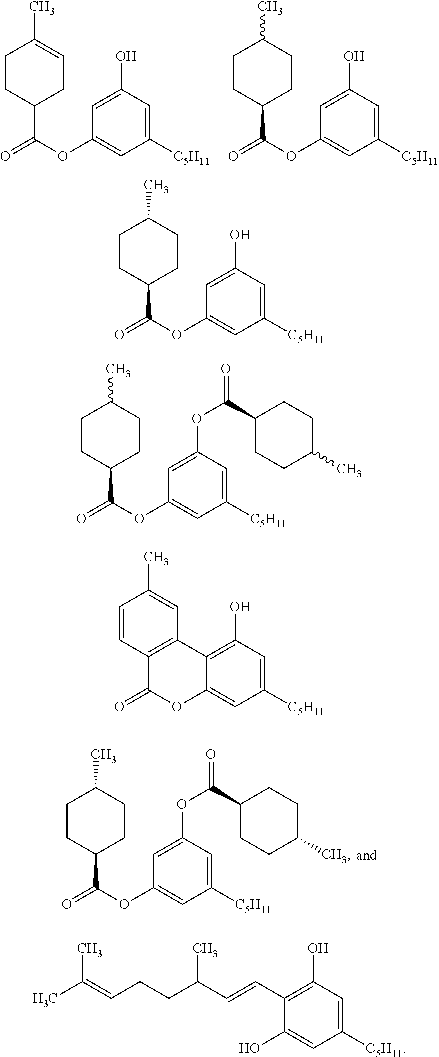

11. The macroreticular polymer beads of any one of claims 1-10, wherein the target cannabinoid is selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), tetrahydrocannabinolic acid A (THCA-A), tetrahydrocannabinolic acid B (THCA-B), tetrahydrocannabinolic acid (THCA), tetrahydrocannabinol (THC), tetrahydrocannabinolic acid C₄ (THCA-C₄), tetrahydrocannabinol C₄ (THC-C₄), tetrahydrocannabivarinic acid (THCVA), tetrahydrocannabivarin (THCV), tetrahydrocannabiorcolic acid (THCA-C₁), tetrahydrocannabiorcol (THC-C₁), Δ⁷-cis-iso-tetrahydrocannabivarin, Δ⁸-tetrahydrocannabinolic acid (Δ8-THCA), cannabivarinodiolic (CBNDVA), cannabivarinodiol (CBNDV), Δ⁸-tetrahydrocannabinol (Δ⁸-THC), Δ⁹-tetrahydrocannabinol (Δ⁹-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabivarinselsoin (CBEV), cannabivarinselsoinic acid (CBEVA), cannabielsoic acid (CBEA), cannabielvarinsoin (CBLV), cannabielvarinsoinic acid (CBLVA), cannabinolic acid (CBNA), cannabinol (CBN), cannabivarinic acid (CBNVA), cannabinol methylether (CBNM), cannabinol-C₄ (CBN-C₄), cannabivarin (CBV), cannabino-C₂ (CBN-C₂), cannabiorcol (CBN-C₁), cannabinodiol (CBND), cannabinodiolic acid (CBNDA), cannabinodivarin (CBDV), cannabitriol (CBT), 10-ethoxy-9-hydroxy-Δ^{6a}-tetrahydrocannabinol, 8,9-dihydroxy-Δ^{6a(10a)}-tetrahydrocannabinol (8,9-Di-OH-CBT-C₅), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ^{6a(10a)}-tetrahydrocannabinol (MEC), Δ⁹-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, and dodeca-2E,4E-dienoic acid isobutylamide.

12. The macroreticular polymer beads of claim 11, wherein the target cannabinoid is CBD.

13. The macroreticular polymer beads of claim 11, wherein the target cannabinoid is CBDA.

14. The macroreticular polymer beads of claim 11, wherein the target cannabinoid is THC.

15. The macroreticular polymer beads of claim 11, wherein the target cannabinoid is THCA.

16. The macroreticular polymer beads of claim 11, wherein the target cannabinoid is selected from the group consisting of CBG, CBN, CBC, and THCV.

17. The macroreticular polymer beads of any one of claims 1-16, wherein the crosslinking monomer is selected from the group consisting of alkylene glycols and polyalkylene glycol diacrylates, polyalkylene glycol methacrylates, vinyl acrylates, vinyl methacrylates, allyl acrylates or allyl methacrylates, divinylbenzene, diallyldiglycol dicarbonate, diallyl maleate, diallyl fumarate, diallyl itaconate, vinyl esters, the dimethacrylates or diacrylates of bis-phenol A or ethoxylated bis-phenol A, methylene bisacrylamide, polymethylene bisacrylamide, bismuth-acrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methyl-2-isocyanatoethyl methacrylate, 1, 1-dimethy 1-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, divinyl benzene; 1,3-divinyltetramethyl disiloxane; 8,13-divinyl-3,7,12,17-tetramethyl-21H,23H-porphine; 8,13-divinyl-3,7,12, 17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; and divinyl tin dichloride.

18. The macroreticular polymer beads of claim 17 wherein the crosslinking monomer is divinylbenzene.

19. The macroreticular polymer beads of any one of claims 1-18, wherein the non-crosslinking monomer is selected from the group consisting of methylmethacrylate, other alkyl methacrylates, alkylacrylates, allyl or aryl acrylates and methacrylates, cyanoacrylate, styrene, substituted styrenes, methyl styrene (multisubstituted) including 1-methylstyrene; 3-methylstyrene; 4-methylstyrene, etc.; vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy) ethyl methacrylate; 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; aryloyl chloride; l-α-acryloyloxy-β,β-dimethyl-γ-butyrolactone; N-acryloxy succinimide acryloxytris(hydroxymethyl)amino-methane; N-acryloyl chloride; N-acryloyl pyrrolidinone; N-acryloyl-tris(hydroxymethyl)amino methane; 2-aminoethyl methacrylate; N-(3-aminopropyl)methacrylamide; (o, m, or p)-amino-styrene; t-amyl methacrylate; 2-(1-aziridinyl)ethyl methacrylate; 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; -bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; t-butylacrylamide; butyl acrylate; butyl methacrylate; (o, m, p)-bromo styrene; t-butyl acrylate; 1-carvone; (S)-carvone; (−)-carvyl acetate; 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 1-(3-butenyl)-4-vinylbenzene; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethy 1-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); glycidyl acrylate, glycidyl methacrylate; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; (±)-linalool; linalyl acetate; p-mentha-1,8-diene; p-mentha-6,8-dien-2-ol; methyleneamino acetonitrile; methacrolein; [3-(methacryloylamino)-propyl] trimethylammonium chloride; methacrylamide; methacrylic acid; methacrylic anhydride; methacrylonitrile; methacryloyl chloride; 2-(methacryloyloxy)ethyl acetoacetate; (3-meth-acryloxypropyl)trimethoxy silane; 2-(methacryloxy)ethyl trimethylammonium methylsulfate; 2-methoxy propene (isopropenyl methyl ether); methyl-2-(bromomethyl)acrylate; 5-methyl-5-hexen-2-one; methyl methacrylate; N,N′methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; methyl vinyl ether; methyl-2-vinyloxirane; methyl vinyl sulfone; 4-methyl-5-vinylthiazole; myrcene; t-nitrostyrene; 3-nitrostyrene; 1-nonadecene; 1,8-nonadiene; 1-octadecene; 1, 7-octadiene; 7-105ctane-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyanoethylene; trans 3-chloroacrylic acid; 2-trifluoromethyl propene; 2-(trifluoromethyl)propenoic acid; 2,4,4′-trimethyl-1-pentene; 3, 5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethylsulfone; 4-(vinyl benzyl)-2-chloroethyl sulfone; N-(p-vinylbenzyl)-N,N′-dimethyl amine; 4-vinyl biphenyl (4-phenylstyrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl peroxcyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinylquinoline; vinyl iodide; vinyllaurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinylquinoline; 1-vinyl silatrane; vinyl sulfone; vinyl sulfonic acid sodium salt; a-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy) silane; vinyl 2-valerate; vinyl benzoic acid; vinyl imidazole, vinylpyridine; vinylbenzylamine; Hydroxyethylmethacrylate (HEMA); and aminopropylmethacrylate.

20. The macroreticular polymer beads of claim 19, wherein the non-crosslinking monomer is styrene.

21. The macroreticular polymer beads of any one of claims 1-20, wherein the non-crosslinking monomer is styrene and the crosslinking monomer is divinylbenzene.

22. The macroreticular polymer beads of any one of claims 1-21, wherein the beads have a selectivity coefficient for the target cannabinoid of greater than about 10.

23. The macroreticular polymer beads of any one of claims 1-22, having a surface area of about 0.1-500 m²/g.

24. The macroreticular polymer beads of any one of claims 1-23, having an average particle size ranging from about 250 μm to about 1.5 mm.

25. A method of preparing macroreticular molecularly imprinted polymer beads comprising:
(1) polymerizing:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid and
(2) removing the cannabinoid surrogate to provide a complexing cavity that selectively binds a target cannabinoid.

26. A method of preparing a macroreticular molecularly imprinted polymer that selectively binds a target cannabinoid, the method comprising:
(1) polymerizing:
(a) a cannabinoid surrogate-containing monomer,
(b) a non-crosslinking monomer, and
(c) a crosslinking monomer;
wherein the cannabinoid surrogate has substantially the same steric and functional properties of the target cannabinoid.

27. The method of any one of claims 25-26, wherein the cannabinoid surrogate-containing monomer has the formula: 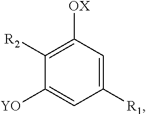
wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, benzoyl, 4-vinylbenzoyl, 2-vinylbenzoyl, 3-vinylbenzoyl, allyl, methacryloyl, acryloyl, carbamoyl, glycidyl methacrylate, and glycidyl acrylate;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid and
wherein at least one of X and Y contains a polymerizable group.

28. The method of claim 27, wherein R₂ is the cannabinoidyl group has the formula:

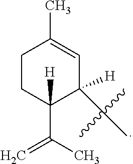

29. The method of claim 27, wherein the cannabinoid surrogate-containing monomer has the formula: 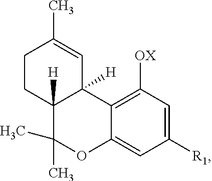
wherein
X contains a polymerizable group.

30. The method of any one of claims 25-29, wherein the polymerizable group is selected from the group consisting of alkenyl, 4-vinylbenzoyl, 2-vinylbenzoyl, 3-vinylbenzoyl, allyl, methacryloyl, acryloyl, carbamoyl, glycidyl methacrylate, and glycidyl acrylate.

31. The method of any one of claims 25-26, wherein the cannabinoid surrogate-containing monomer is selected from the group consisting of:

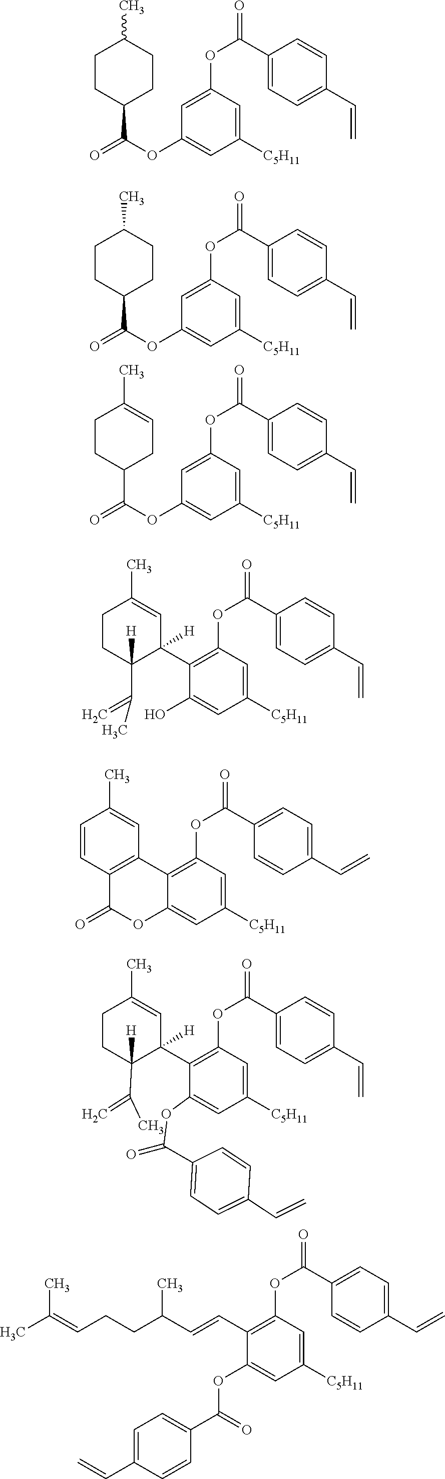

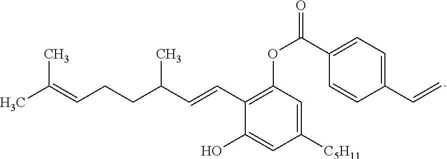

32. A method of preparing macroreticular molecularly imprinted polymer beads, the method comprising:
(1) polymerizing:
(a) cannabinoid surrogate,
(b) a non-crosslinking monomer, and
(c) a crosslinking monomer:
wherein the cannabinoid surrogate has substantially the same steric and functional properties of the target cannabinoid and
(2) removing the cannabinoid surrogate to provide a complexing cavity that selectively binds a target cannabinoid.

33. The method of claim 32, wherein the cannabinoid surrogate has the formula: 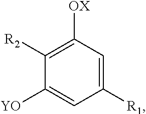
wherein
R₁ is selected from the group consisting of H, alkyl, acyl, and aryl;
R₂ is selected from the group consisting of H, a cannabinoidyl group, alkyl, aryl, alkenyl, and acyl;
X is selected from the group consisting of H, alkyl, acyl, aryl, polyether, alkenyl, and benzoyl;
Y is selected from the group consisting of H, a cannabinoidyl group, alkyl, aryl, polyether, alkenyl, benzoyl, 4-vinylbenzoyl, 2-vinylbenzoyl, 3-vinylbenzoyl, allyl, methacryloyl, substituted methacryloyl, acryloyl, carbamoyl, 2-hydroxyethyl methacrylate, 2-hydroxyethyl acrylate, glycidyl methacrylate, and glycidyl acrylate; or
Y and R₂ together with the atoms to which they are attached form a cannabinoidyl group.
wherein the cannabinoid surrogate has substantially the same steric and functional properties of a target cannabinoid.

34. The method of claim 32, wherein the cannabinoid surrogate is selected from the group consisting of:

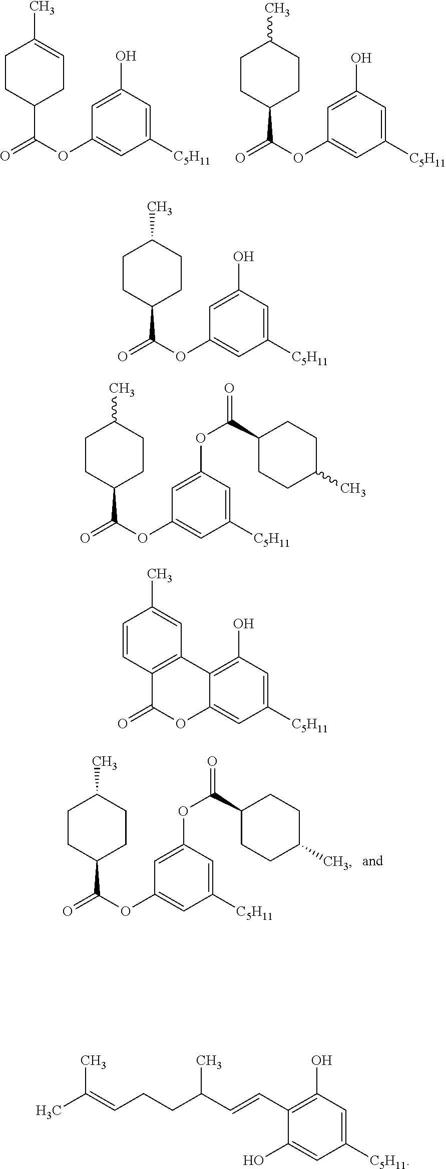

35. The method of any one of claims 25-34, wherein the non-crosslinking monomer is styrene.

36. The method of any one of claims 25-35, wherein the crosslinking monomer is divinylbenzene.

37. The method of any one of claims 25-36, wherein the non-crosslinking monomer is styrene and the crosslinking monomer is divinylbenzene.

38. The method of any one of claims 25-37, wherein polymerizing occurs in the presence of a pore forming additive.

39. The macroreticular molecularly imprinted polymer beads prepared according to any one of claims 25 and 27-38.

40. The macroreticular molecularly imprinted polymer prepared according to any one of claims 26-38.

41. A method of selectively sequestering one or more target cannabinoids from a solution of one or more target cannabinoids admixed with other cannabinoids, the method comprising:
first contacting the macroreticular polymer beads of any one of claims 1 and 3-24 with a hydrolyzing solution, whereby the cannabinoid surrogates are removed from the macroreticular polymer beads, then contacting the stripped beads with the cannabinoid-containing solution, thereby selectively sequestering the target cannabinoid in the macroreticular polymer beads.

42. A method of selectively sequestering one or more target cannabinoids from a solution of one or more target cannabinoids admixed with other cannabinoids, the method comprising:
contacting the macroreticular molecularly imprinted polymer of any one of claims 2-24 with the cannabinoid-containing solution, thereby selectively sequestering the target cannabinoid in the macroreticular polymer beads.

43. The method of any one of claims 41-42, further comprising stripping the target cannabinoid from the macroreticular polymer beads.

44. A method of removing THC from a cannabinoid-containing solution comprising THC and CBD, the method comprising:
(1) contacting the solution with the macroreticular polymer beads of any one of claims 1-24, whereby the THC is selectively complexed in the beads, and
(2) recovering a solution comprising CBD that is substantially THC-free.

45. The method of claim 44, further comprising stripping the beads with a stripping solution, whereby the THC is substantially removed from the beads.

46. A method of removing at least two target cannabinoids from a solution containing at least two target cannabinoids admixed with other cannabinoids, the method comprising:
(1) providing an activated form of the macroreticular polymer beads of any one of claims 1, and 3-24 having an affinity for a first target cannabinoid;
(2) contacting the solution with the macroreticular polymer beads, whereby the first target cannabinoid is selectively complexed in the beads,
(3) recovering a solution that is substantially free of the first target cannabinoid; and
(4) providing an activated form of the macroreticular polymer beads of any one of claims 1, and 3-24, having an affinity for a second target cannabinoid in the recovered solution;
(5) contacting the solution of Step (3) with the macroreticular polymer beads, whereby the second target cannabinoid is selectively complexed in the beads, and
(6) recovering a solution that is substantially free of the first and second target cannabinoids.

47. A method of removing at least two target cannabinoids from a solution containing at least two target cannabinoids admixed with other cannabinoids, the method comprising:
(1) providing the macroreticular polymer beads of any one of claims 2-24 having an affinity for a first target cannabinoid;
(2) contacting the solution with the macroreticular polymer beads, whereby the first target cannabinoid is selectively complexed in the macroreticular molecularly imprinted polymer beads,
(3) recovering a solution that is substantially free of the first target cannabinoid; and
(4) providing the macroreticular molecularly imprinted polymer beads of any one of claims 2-24, having an affinity for a second target cannabinoid in the recovered solution;
(5) contacting the solution of Step (3) with the macroreticular polymer beads, whereby the second target cannabinoid is selectively complexed in the beads, and
(6) recovering a solution that is substantially free of the first and second target cannabinoids.

48. The method of claim 46 or 47, further comprising repeating the method until substantially all target cannabinoids are removed.

49. The method of claim 48, further comprises repeating the method 1-10 more times.

50. The method of any one of claims 46-49, wherein the target cannabinoid is selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), tetrahydrocannabinolic acid A (THCA-A), tetrahydrocannabinolic acid B (THCA-B), tetrahydrocannabinolic acid (THCA), tetrahydrocannabinol (THC), tetrahydrocannabinolic acid C₄ (THCA-C₄), tetrahydrocannabinol C₄ (THC-C₄), tetrahydrocannabivarinic acid (THCVA), tetrahydrocannabivarin (THCV), tetrahydrocannabiorcolic acid (THCA-C₁), tetrahydrocannabiorcol (THC-C₁), Δ⁷-cis-iso-tetrahydrocannabivarin, Δ⁸-tetrahydrocannabinolic acid (Δ8-THCA), cannabivarinodiolic (CBNDVA), cannabivarinodiol (CBNDV), Δ⁸-tetrahydrocannabinol (Δ⁸-THC), Δ⁹-tetrahydrocannabinol (Δ⁹-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabivarinselsoin (CBEV), cannabivarinselsoinic acid (CBEVA), cannabielsoic acid (CBEA), cannabielvarinsoin (CBLV), cannabielvarinsoinic acid (CBLVA), cannabinolic acid (CBNA), cannabinol (CBN), cannabivarinic acid (CBNVA), cannabinol methylether (CBNM), cannabinol-C₄ (CBN-C₄), cannabivarin (CBV), cannabino-C₂ (CBN-C₂), cannabiorcol (CBN-C₁), cannabinodiol (CBND), cannabinodiolic acid (CBNDA), cannabinodivarin (CBDV), cannabitriol (CBT), 10-ethoxy-9-hydroxy-Δ^{6a}-tetrahydrocannabinol, 8,9-dihydroxy-Δ^{6a(10a)}-tetrahydrocannabinol (8,9-Di-OH-CBT-C₅) cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-Δ^{6a(10a)}-tetrahydrocannabinol (MEC), Δ⁹-cis-tetrahydrocannabinol (cis-THC), cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), yangonin, epigallocatechin gallate, dodeca-2E, 4E, 8Z, 10Z-tetraenoic acid isobutylamide, and dodeca-2E,4E-dienoic acid isobutylamide.

51. The method of claim 50, wherein the target cannabinoids are THC and CBD.

52. A method of removing THC from a cannabinoid-containing solution comprising THC and CBD, the method comprising:
(1) contacting the solution with the macroreticular polymer beads of any one of claims 1-24, whereby the THC and CBD are complexed in the beads;
(2) selectively stripping CBD from the macroreticular polymer beads; and
(2) recovering a stripped solution comprising CBD that is substantially THC-free.

53. A method of purifying a cannabinoid-containing solution, the method comprising:
(1) contacting the solution with the macroreticular polymer beads of any one of claims 1-24, whereby the cannabinoids in the solution are complexed in the beads and
(2) stripping the cannabinoids from the macroreticular polymer beads to provide a purified cannabinoid-containing solution.

54. The method of claim 53, wherein the purified cannabinoid-containing solution contains less than about 0.1 ppm pesticides.

55. The method of any one of claims 53-54, wherein the purified cannabinoid-containing solution contains less than about 10 ppm of heavy metals.

56. The method of any one of claims 41-55, wherein the solution is a Cannabis or hemp extract.

57. The method of claim 56, wherein the Cannabis or hemp plant extract components are semi-processed.

58. The method of any one of claims 41-57 wherein the solution is prepared by:
i) providing cannabinoid containing plant trim;
ii) extracting the plant trim with an extraction solvent to provide an extract; and
iii) optionally winterizing and/or filtering and/or distilling the extract to provide the solution.

59. The method of claim 58, wherein the extraction solvent is an alcohol/water mixture.

60. The method of any one of claims 46-59, further comprising stripping the target cannabinoid complexed beads or polymer with a stripping solution, whereby the target cannabinoid is substantially removed from the beads.

61. The method of claim 43, 45, or 60, wherein the stripping solution is an alcohol or an alcohol/water mixture.

62. The method of claim 61, wherein the stripping solution is 50-70% alcohol/water.

63. The method of any one of claims 59-62, wherein the alcohol is selected from the group consisting of methanol, ethanol and isopropanol.

64. The method of claim 43, 45, or 60, wherein stripping provides substantially pure target cannabinoid.

65. The method of claim 64, wherein the target cannabinoid is CBD and stripping provides a substantially pure CBD comprising <1% THC w/w.

66. The method of claim 65, wherein stripping provides a substantially pure CBD comprising ≤0.3% THC w/w.

67. The method of claim 65 or 66, wherein stripping provides a substantially pure CBD comprising <0.09%% THC w/w.

68. A compound selected from the group consisting of: 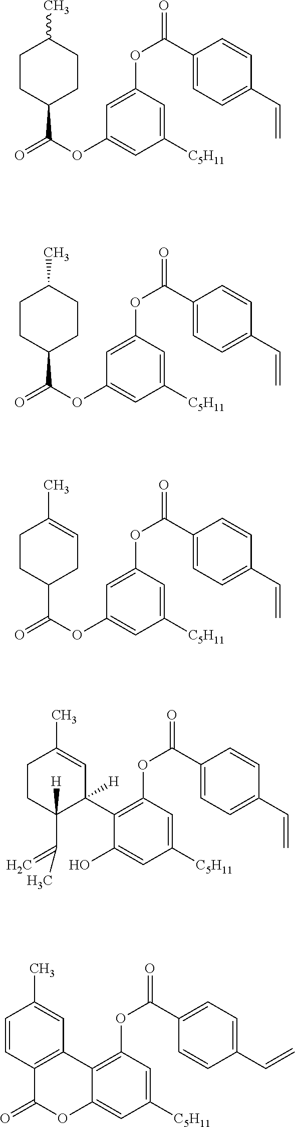 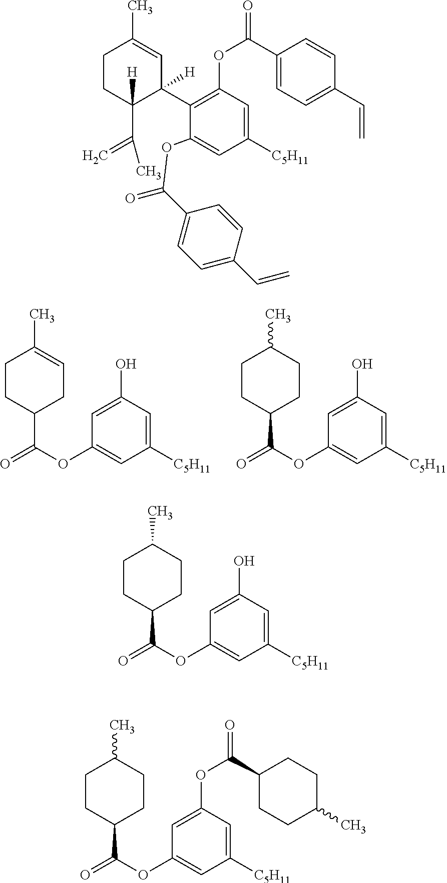 [chemical structure]
